(19) United States
(12) Patent Application Publication
(10) Pub. No.: US 2002/0037567 A1
(43) Pub. Date: Mar. 28, 2002

(54) Alpha(III) subunit for prolyl 4-hydroxylase

(76) Inventors: Kari Kivirikko, Oulu (FI); Johanna Myllyharju, Oulu (FI); Liisa Kukkola, Oulu (FI); Reija Hieta, Oulu (FI)

Correspondence Address: ROBINS & PASTERNAK LLP, 90 MIDDLEFIELD ROAD, SUITE 200, MENLO PARK, CA, 94025, US

(21) Appl. No.: 09/809,342

(22) Filed: Jun. 4, 2001

Related U.S. Application Data
(60) Provisional application No. 60/189,373, filed on Mar. 15, 2000.

(51) Int. Cl. A61K 31/00; C12P 21/02; C12N 5/06; C12Q 1/68; C07H 21/04; C12N 9/02

(52) U.S. Cl. 435/189; 435/69.1; 435/6; 435/325; 514/1; 536/23.2; 530/388.1

(57) ABSTRACT

The present invention relates to new isoforms of the alpha subunit of prolyl 4-hydroxylase, encoding polynucleotides, and related methods of production and use.

### CROSS-REFERENCE TO RELATED APPLICATION

This application is related to provisional patent application Ser. No. 60/189,373, filed Mar. 15, 2000, from which priority is claimed under 35 USC §119(e)(1) and which is incorporated herein by reference in its entirety.

### FIELD OF THE INVENTION

The present invention relates to alpha (“α”) subunits of prolyl 4-hydroxylase and the polynucleotides encoding them, and to methods of making and using these polypeptides, for example, and polynucleotides in the production of recombinant collagen, and in the diagnosis, prevention, and treatment of various diseases and disorders.

More specifically, the present invention relates to polypeptides of a new α subunit of prolyl 4-hydroxylase, designated the “alpha(III) subunit,” and variants thereof, to the encoding polynucleotides, and to various methods of production, diagnosis, treatment, and prevention using these polypeptides and polynucleotides.

### BACKGROUND

Collagens are structural proteins comprised of one or more collagen subunits which together form at least one triple-helical domain. A variety of enzymes are utilized in order to transform collagen subunits into procollagen or other precursor molecules and then into mature collagen. Such enzymes include prolyl-4-hydroxylase, C-proteinase, N-proteinase, lysyl oxidase and lysyl hydroxylase.

Prolyl 4-hydroxylase plays a crucial role in the synthesis of all collagens. Specifically, the enzyme catalyzes the formation of 4-hydroxyproline in collagens and related proteins by the hydroxylation of proline residues in -Xaa-Pro-Bly-sequences. These 4-hydroxyproline residues are essential for the folding of newly synthesized collagen polypeptide chains into triple-helical molecules. Vertebrate prolyl 4-hydroxylase is an α₂β₂ tetramer in which the a subunits contribute to most parts of the catalytic sites. (See, e.g., Kivirikko et al. (1989) FASEB J. 3:1609-1617; Kivirikko et al. (1990) Ann. N.Y. Acad. Sci. 580:132-142; Kivirikko et al. (1992), Post Translational Modifications of Proteins, eds. Harding, J. J. and M. J. C. Crabbe, CRC, Boca Raton, Fla., pp.1-51.) The beta (“β”) subunit has been cloned from many sources and has been found to be a highly unusual multifunctional polypeptide identical to the enzyme protein disulfide-isomerase, a cellular thyroid hormone-binding protein, the smaller subunit of the microsomal triacylglycerol transfer protein, and an endoplasmic reticulum luminal polypeptide which uniquely binds to various peptides. (See, e.g., Pihlajaniemi et al. (1987) EMBO J. 6:643-649; Kojvu et al. (1987) J. Biol. Chem. 262:6447-49; Cheng et al. (1987) J. Biol. Chem. 262:11221-11227; Wetterau et al. (1990) J. Biol. Chem. 265:9800-9807; Noiva et al. (1991) J. Biol. Chem. 266:19645-19649; Noiva et al. (1993) J. Biol. Chem. 268:19210-19217; Noiva and Lennatz (1992) J. Biol. Chem. 267:6447-49; Freedman et al. (1994) Trends Biochem. Sci. 19:331-336.)

A catalytically important alpha subunit, designated the alpha(I) subunit, has been cloned from human (Helaakoski et al. (1989) Proc. Natl. Acad. Sci. (USA) 86:4392-4396), chicken (Bassuk et al. (1989) Proc. Natl. Acad. Sci. (USA) 86:7382-7886), and Caenorhabditis elegans (Veijola et al. (1994) J. Biol. Chem. 269:26746-26753), and its RNA transcripts have been shown to undergo alternative splicing involving sequences encoded by two consecutive, homologous 71-bp exons (Helaakoski, supra; Helaakoski et al. (1994) J. Biol. Chem. 269:27847-27854). A second alpha subunit, designated the alpha(II) subunit, has been previously obtained from mouse and from human. (See, e.g., Helaakoski et al. (1995) Proc. Natl. Acad. Sci. (USA) 92:4427-4431 and U.S. Pat. No. 5,928,922, both incorporated herein in their entirety.)

### SUMMARY

In one aspect, the invention includes a polypeptide comprising the amino acid sequence of SEQ ID NO:2; variants of SEQ ID NO:2 and fragments of SEQ ID NO:2.

In another aspect, the invention includes a polynucleotide encoding any of the polypeptides described herein. Preferably, the polypeptide exhibits one or more of the characteristics of full-length alpha(III). Also provided are polynucleotides (or fragments thereof) that exhibit between 80 and 100% (or any integer therebetween) sequence identity to the sequence shown in SEQ ID NO:1. In certain embodiments, the polynucleotides are at least 80% identical, when optimally aligned, to the sequence shown in SEQ ID NO:1. In other embodiments, the polynucleotides are at least 90% identical, when optimally aligned, to the sequence shown in SEQ ID NO:1. In yet other aspects, an isolated and purified polynucleotide which hybridizes under stringent conditions to any of the polynucleotides described herein is provided. Also provided is an isolated and purified polynucleotide which is complementary to any of the polynucleotides described herein.

In another aspect, the invention includes an expression vector comprising any of the polynucleotides described herein. In certain embodiments, the expression vector further comprises a nucleotide sequence encoding a beta (“β”) subunit of prolyl 4-hydroxylase. In addition, any of the expression vectors described herein can further comprise a nucleotide sequence encoding one or more collagen and/or procollagen molecules of any type.

Host cells comprising one or more of the polynucleotides and/or one or more of the expression vectors described herein form another aspect of the invention. In certain embodiments, the host cells further comprise one or more polynucleotide sequences encoding a beta subunit of prolyl 4-hydroxylase and/or one or more nucleotide sequences encoding one or more collagen molecules. Any suitable host cell can be used, for example eukaryotic cells (e.g., mammalian, insect, plant, yeast, etc.) or prokaryotic cells (e.g., bacteria).

In another aspect, the invention provides a method for producing a polypeptide, the method comprising: culturing any of the host cells described herein under conditions suitable for expression of the polypeptide; and (b) isolating the polypeptide.

In yet another aspect, a method for producing a prolyl 4-hydroxylase tetramer is provided, the method comprising: (a) culturing any of the host cells described herein under conditions suitable for formation of the prolyl 4-hydroxylase tetramer; and (b) recovering the prolyl 4-hydroxylase tetramer.

In a still further aspect, the invention includes a method for detecting a polynucleotide in a sample, the method comprising: (a) hybridizing any of the polynucleotides described herein to at least one nucleic acid in a sample, thereby forming a hybridization complex; and (b) detecting the hybridization complex, wherein the presence of the hybridization complex is indicative of the presence of the polynucleotide in the sample.

In a still further aspect, pharmaceutical compositions comprising any of the polypeptides and/or polynucleotides described herein and a suitable pharmaceutical carrier are provided.

Antibodies that specifically bind to any of the polypeptides described herein are also provided as are agonists and antagonists of any of these polypeptides.

In another aspect, the invention includes a method for treating or preventing a disorder associated with decreased expression or activity of the alpha(III) subunit of prolyl 4-hydroxylase, the method comprising administering to a subject in need an effective amount of any of the pharmaceutical compositions, antagonists and/or agonists described herein.

In another aspect, the invention includes a method of aiding in the diagnosis of a condition associated with altered expression of an alpha(III) subunit of prolyl 4-hydroxylase, comprising (a) detecting the expression of an alpha(III) subunit gene in a test sample; and (b) comparing result of step (a) to expression levels of the alpha(III) subunit gene in a control cell, wherein altered of the alpha(III)subunit gene in the test sample is indicative of the condition.

A solid phase support comprising any of the polynucleotides, polypeptides and/or antibodies described herein is also provided.

These and other embodiments of the present invention will readily occur to those of ordinary skill in the art in view of the disclosure herein.

### BRIEF DESCRIPTION OF THE FIGURES

FIG. 1 shows a polypeptide and a polynucleotide sequence corresponding to the human alpha(III) subunit of prolyl 4-hydroxylase of the present invention.

FIG. 2 shows the results of an RT-PCR assay showing that the human alpha(III) subunit of prolyl 4-hydroxylase of the present invention is significantly expressed in fetal tissues.

FIG. 3 shows the results of an RT-PCR assay performed with a Human Fetal Multiple Tissue cDNA Panel (Clontech Laboratories Inc., Palo Alto, Calif.), showing that the human alpha(III) subunit of prolyl 4-hydroxylase of the present invention is significantly expressed in fetal tissues.

FIG. 4 shows the nucleotide sequence (SEQ ID NO:1) corresponding to the coding region of the human alpha(III) subunit of prolyl-4-hydroxylase.

FIG. 5 shows the amino acid sequence (SEQ ID NO:2) of the human alpha(III) subunit of prolyl-4-hydroxylase encoded by the nucleotide sequence of SEQ ID NO:1.

FIG. 6 shows an alignment of a human alpha(III) subunit described herein with human alpha(I) and alpha(II) subunits. The Figure depicts a “CLUSTAL W (1.81) Multiple Sequence Alignment” in Pearson format. The top line (alpha1) shows a 517 amino acid sequence of an alpha(I) subunit. The middle line (alpha2) shows a 514 amino acid sequence of an alpha(II) subunit and the bottom line (alpha3) shows a 544 amino acid sequence (SEQ ID NO:2) of an alpha(III) subunit. Alignment scores were as follows: alpha1: alpha2, score: 62; alpha2 to alpha3, score: 37; and alpha1 to alpha3, score 35.

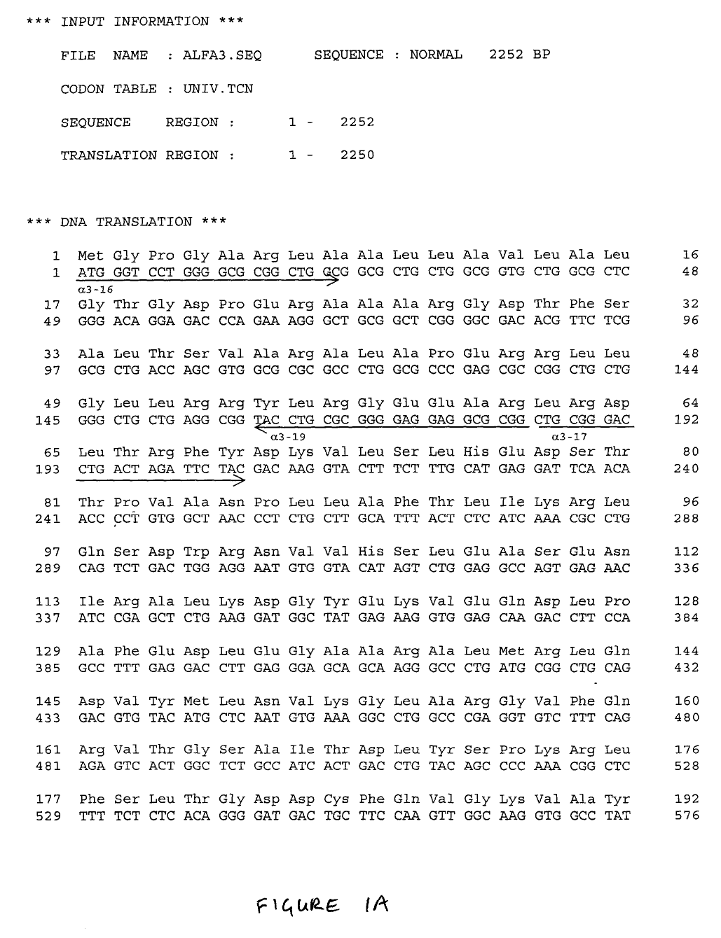

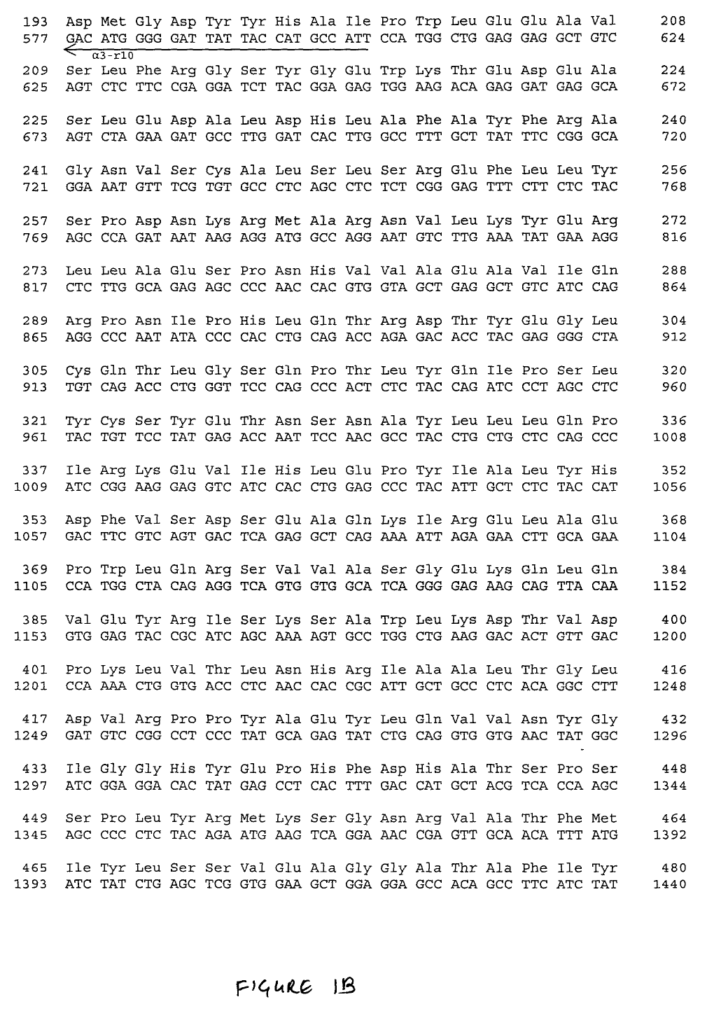

### DETAILED DESCRIPTION

Before the present polypeptides, polynucleotides, and related methods are described, it is to be understood that the invention is not limited to the particular methodologies, protocols, cell lines, vectors, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a host cell” includes a plurality of such host cells, a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications which might be used in connection with the invention. Nothing herein is to be construed as an admission that the invention is not entitled to antedate such disclosure by virtue of prior invention.

As used herein, the term “antibody” refers to intact molecules as well as fragments thereof, such as Fab, F(ab′)₂, and Fᵥ, which are capable of binding the epitopic determinant. Antibodies that bind the polypeptides of the present invention can be prepared using intact polypeptides or fragments containing small peptides of interest as the immunizing antigen. The polypeptide or oligopeptide used to immunize an animal can be derived from the translation of RNA or synthesized chemically and can be conjugated to a carrier protein, if desired. Commonly used carriers that are chemically coupled to peptides include bovine serum albumin and thyroglobulin, keyhole limpet hemocyanin. The coupled peptide is then used to immunize the animal (e.g., a mouse, a rat, or a rabbit).

The term “humanized antibody”, as used herein, refers to antibody molecules in which amino acids have been replaced in the non-antigen binding regions in order to more closely resemble a human antibody, while still retaining the original binding ability.

The term “sample” is used herein in its broadest sense. Samples may be derived from any source, for example, from bodily fluids, secretions, or tissues including, but not limited to, saliva, blood, urine, and organ tissue (e.g., biopsied tissue); from chromosomes, organelles, or other membranes isolated from a cell; from genomic DNA, RNA, or cDNA in solution or bound to a substrate; and from cleared cells or tissues, or blots or imprints from such cells or tissues. Methods for obtaining such samples are within the level of skill in the art. A sample can be in solution or can be, for example, fixed or bound to a substrate. A sample can refer to any material suitable for testing for the presence of the polypeptides or of mRNA corresponding to the polypeptides of the present invention or suitable for screening for molecules that bind to the polypeptides or fragments thereof. Methods for obtaining such samples are within the level of skill in the art.

An “antisense sequence” is any sequence capable of specifically hybridizing to a target sequence. The antisense sequence can be DNA, RNA, or any nucleic acid mimic or analog. The term “antisense technology” refers to any technology which relies on the specific hybridization of an antisense sequence to a target sequence.

The term “subunit” refers to a polypeptide that forms a component of a multi-component protein. The various subunits of a given protein (e.g., an enzyme such as prolyl 4-hydroxylase) can be encoded by a single gene, as well as any derivatives of that polypeptide sequence, including deletions, additions, derivatives, conservative substitutions, etc.

The term “hybridization” refers to the process by which a nucleic acid sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency. In particular, stringency can be increased by reducing the concentration of salt, increasing the concentration of formamide, or raising the hybridization temperature.

For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 μg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. To remove nonspecific signals, blots can be sequentially washed, for example, at room temperature under increasingly stringent conditions of up to 0.1×saline sodium citrate and 0.5% sodium dodecyl sulfate. Variations on the above ranges and conditions are well known in the art.

“Polypeptide” as used herein refers to an amino acid, oligopeptide, peptide, polypeptide, or protein sequence, and fragment thereof, and to naturally occurring or synthetic molecules. “Fragments” can refer to any portion of a full-length amino acid sequence which retains at least one structural or functional characteristic of the protein.

The terms “complementary” or “complementarity”, as used herein, refer to the natural binding of polynucleotides by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of PNA molecules.

A “deletion”, as used herein, refers to a change in the amino acid or nucleotide sequence and results in the absence of one or more amino acid residues or nucleotides.

An “insertion” or “addition”, as used herein, refers to a change in an amino acid or nucleotide sequence resulting in the addition of one or more amino acid residues or nucleotides, respectively, as compared to the naturally occurring molecule.

The term “polynucleotide” refers to a nucleic acid, oligonucleotide, nucleotide, or polynucleotide, sequence fragments thereof, and to DNA or RNA of genomic or synthetic origin which may be single- or double-stranded and may represent the sense or antisense strand, to peptide nucleic acid (PNA), or to any DNA-like or RNA-like material, natural or synthetic in origin.

An “expression cassette” comprises any nucleic acid construct which contains polynucleotide gene(s) or sequence(s) capable of being expressed in a cell. Expression cassettes may contain, in addition to polynucleotide gene(s) or sequence(s) of interest, additional transcriptional, translational or other regulatory or control elements. Such cassettes are typically constructed into a “vector,” “vector construct,” or “expression vector,” (i.e., a “nucleic acid expression vector”) in order to transfer the expression cassette into target cells.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transformed. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

“Fragments” refers to nucleic acid sequences which are between 5 and 10,000 nucleotides in length. For certain applications (e.g., as primers), fragments are preferably between about 5 and 500 nucleotides in length (or any integer value therebetween), more preferably between about 10 and 100 nucleotides in length (or any integer value therebetween), even more preferably 10 to 50 nucleotides in length (or any integer value therebetween). For other applications (e.g., probes), fragments are preferably greater than 50 nucleotides than in length, and which most preferably are at least 100 nucleotides, 1000 nucleotides, or at least 10,000 nucleotides in length (or any integer value between 60 and 10,000 nucleotides). Similarly, “fragments” of a protein or polypeptide refers to any length amino acid sequence that is not the full-length. Fragments of the polypeptides are typically greater than 3 to 5 amino acids in length, preferably greater than 10 amino acids in length, more preferably at least about 25 amino acids in length, more preferably at least about 50 amino acids in length and even more preferably at least about 100 amino acids in length.

A “substitution”, as used herein, refers to the replacement of one or more amino acids or nucleotides by different amino acids or nucleotides, respectively.

A “variant,” as the term is used herein, is an amino acid sequence that is altered by one or more amino acids. The variant may have conservative changes, wherein a substituted amino acid has similar structural or chemical properties, e.g., replacement of leucine with isoleucine. More rarely, a variant may have nonconservative changes, e.g., replacement of a glycine with a tryptophan. Analogous minor variations may also include amino acid deletions or insertions, or both. The term “variant” can also be used in reference to nucleotide sequences that are altered by one or more nucleotides. The altered nucleotide(s) may or may not result in changes to the resulting polypeptide. Guidance in determining which amino acid or nucleotide residues may be substituted, inserted, or deleted may be found using computer programs well known in the art, for example, DNASTAR software (DNASTAR Inc., Madison, Wis.).

The phrases “% similarity” or “% identity” refer to the percentage of sequence similarity or identity found in a comparison of two or more amino acid or nucleic acid sequences. Percent similarity can be determined by methods well-known in the art. For example, percent similarity between amino acid sequences can be calculated using the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent similarity can be calculated by other methods known in the art, for example, by varying hybridization conditions, and can be calculated electronically using programs such as the MEGALIGN program. (DNASTAR Inc., Madison, Wis.)

The term “purified” as used in reference to prolyl 4-hydroxylase molecules (e.g., polynucleotides or polypeptides) denotes that the indicated molecules are present in the substantial absence of other biological macromolecules, e.g., polynucleotides, proteins, and the like. The term “purified” as used herein preferably means at least 95% by weight, more preferably at least 99.8% by weight, of the indicated biological macromolecules present (but water, buffers, and other small molecules, especially molecules having a molecular weight of less than 1000 daltons can be present).

The term “substantially purified”, as used herein, refers to nucleic or amino acid sequences that are removed from their natural environment, isolated or separated, and are at least 60% free, preferably 75% free, and most preferably 90% free from other components with which they are naturally associated.

The term “isolated” as used herein refers to a nucleic acid or protein molecule separated not only from other nucleic acids or proteins that are present in the source material, but also from other nucleic acids and proteins, and preferably refers to a nucleic acid or protein molecule found in the presence of (if anything) only a solvent, buffer, ion, or other component normally present in a solution of the same. The terms “isolated” and “purified” do not encompass molecules present in their natural source.

General Overview

The present invention describes novel polynucleotide sequences encoding an alpha(III) subunit of prolyl 4-hydroxylase. Polypeptides encoded by these polynucleotides and antibodies directed against these polypeptides (or fragments thereof) are also described. Methods of making and using these polynucleotides, polypeptides, antibodies and other molecules, for example in diagnostic, therapeutic or other applications is also contemplated.

Alpha(III) Prolyl 4-Hydroxylase Polynucleotides

In one aspect, the present invention relates to a polynucleotide encoding an alpha(III) subunit of prolyl 4-hydroxlase or a functional equivalent thereof. In one aspect, the polynucleotide comprises the polynucleotide sequence of SEQ ID NO:1. In a further aspect, the isolated nucleic acid molecule comprises a polynucleotide sequence with greater than 70% similarity (e.g., 70-100% or any integer value therebetween) to the polynucleotide sequence of SEQ ID NO:1, more preferably, of greater than 80% (e.g., 80-100% or any integer value therebetween) similarity to the polynucleotide of SEQ ID NO:1, and, most preferably, of greater than 90% (e.g., 90-100% or any integer value therebetween) similarity to the polynucleotide of SEQ ID NO:1.

The invention also encompasses production of polynucleotide sequences, fragments, or complements thereof, encoding the present polypeptides, entirely by synthetic chemistry. After production, the synthetic sequence may be inserted into any of the many available expression vectors and cell systems using reagents that are well known in the art. Moreover, synthetic chemistry may be used to introduce mutations into a polynucleotide sequence encoding a collagen or functional equivalents thereof.

In accordance with the invention, polynucleotide sequences which encode the alpha(III) subunit of SEQ ID NO:2 or any functional equivalent thereof may be used to direct the expression of the alpha(III) subunit in appropriate host cells.

It will be appreciated by those skilled in the art that as a result of the degeneracy of the genetic code, a multitude of polynucleotide sequences encoding the polypeptides of the present invention or functional equivalents thereof, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code.

Altered polynucleotide sequences which may be used in accordance with the invention include deletions, additions or substitutions of different nucleotide residues resulting in a sequence that encodes the same or a functionally equivalent gene product. The gene product itself may contain deletions, additions or substitutions of amino acid residues within an prolyl 4-hydroxylase subunit sequence, which result in a functionally equivalent prolyl 4-hydroxylase subunit. Such amino acid substitutions may be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine.

The polynucleotide sequences of the invention may be engineered in order to alter the prolyl 4-hydroxylase subunit sequence for a variety of ends including, but not limited to, alterations which modify processing and expression of the gene product. For example, alternative secretory signals may be substituted for the native secretory signal and/or mutations may be introduced using techniques which are well known in the art, e.g., site-directed mutagenesis, to insert new restriction sites, to alter glycosylation patterns, phosphorylation, etc. Additionally, when expressing in non-human cells, the polynucleotides encoding the alpha subunits of the present invention of the invention may be modified in the silent position of any triplet amino acid codon so as to better conform to the codon preference of the particular host organism.

In one method, polynucleotides of the present invention that encode an alpha(III) prolyl 4-hydroxylase subunit or fragments or variants thereof are changed via site-directed mutagenesis. This method uses oligonucleotide sequences that encode the polynucleotide sequence of the desired amino acid variant, as well as a sufficient adjacent nucleotide on both sides of the changed amino acid to form a stable duplex on either side of the site of being changed. In general, the techniques of site-directed mutagenesis are well known to those of skill in the art. (See, e.g., Edelman et al. (1983) DNA 2:183.) A versatile and efficient method for producing site-specific changes in a polynucleotide sequence was published by Zoller and Smith (1982) Nucleic Acids Res. 10:6487-6500.

As known in the art, mutations on the nucleotide sequence do not necessarily alter the amino acid sequence encoded by the nucleic acid molecule, but can merely provide unique restriction sites useful for manipulation of the molecule. Thus, the modified molecule can be made up of a number of discrete regions, or D-regions, flanked by unique restriction sites. These discrete regions of the molecule are herein referred to as cassettes. Molecules formed of multiple copies of a cassette are encompassed by the present invention. Recombinant or mutant nucleic acid molecules or cassettes which provide desired characteristics such as resistance to endogenous enzymes are also encompassed by the present invention.

PCR may also be used to create amino acid sequence variants of a collagen. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the collagen at the position specified by the primer. The product DNA fragments replace the corresponding region in the plasmid and this gives the desired amino acid variant. A further technique for generating amino acid variants is the cassette mutagenesis technique described in Wells et al. (1985) Gene 34:315 and other mutagenesis techniques well known in the art, such as, for example, the techniques described generally in Sambrook et al., supra, and Current Protocols in Molecular Biology, Ausubel et al., supra.

In an alternate embodiment of the invention, the coding sequence of the alpha(III) subunit of prolyl 4-hydroxylase of the invention could be synthesized in whole or in part, using chemical methods well known in the art. See, for example, Caruthers et al., Nuc. Acids Res. Symp. Ser. 7:215-233 (1980); Crea and Horn, Nuc. Acids Res. 9(10):2331 (1980); Matteucci and Caruthers, Tetrahedron Letters 21:719 (1980); and Chow and Kempe, Nuc. Acids Res. 9(12):2807-2817 (1981).

Alpha(III) Prolyl 4-Hydroxylase Polypeptides

The present invention is further directed to polypeptides comprising the alpha(III) subunit amino acid sequence of SEQ ID NO:1 or fragments or variants thereof. The present polynucleotides thus include sequences which encode variants of the described alpha(III) subunit of prolyl 4-hydroxylase subunit or fragments or derivatives thereof. These amino acid sequence variants, fragments, and derivatives may be prepared by various methods known in the art for introducing appropriate nucleotide changes into a native or variant polynucleotide encoding the subunit. Two important variables in the construction of amino acid sequence variants are the location of the mutation and the nature of the mutation. The amino acid sequence variants are preferably constructed by mutating the polynucleotide to give an amino acid sequence that does not occur in nature. These amino acid alterations can be made at sites that differ in enzyme subunits from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site.

Amino acids are divided into groups based on the properties of their side chains (polarity, charge, solubility, hydrophobicity, hydrophilicity, and/or the amphipathic nature): (1) hydrophobic (leu, met, ala, ile), (2) neutral hydrophobic (cys, ser, thr), (3) acidic (asp, glu), (4) weakly basic (asn, gln, his), (5) strongly basic (lys, arg), (6) residues that influence chain orientation (gly, pro), and (7) aromatic (trp, tyr, phe). Conservative changes encompass variants of an amino acid position that are within the same group as the “native” amino acid. Moderately conservative changes encompass variants of an amino acid position that are in a group that is closely related to the “native” amino acid (e.g., neutral hydrophobic to weakly basic). Non-conservative changes encompass variants of an amino acid position that are in a group that is distantly related to the “native” amino acid (e.g., hydrophobic to strongly basic or acidic).

Amino acid sequence deletions generally range from about 1 to 30 residues, preferably from about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells.

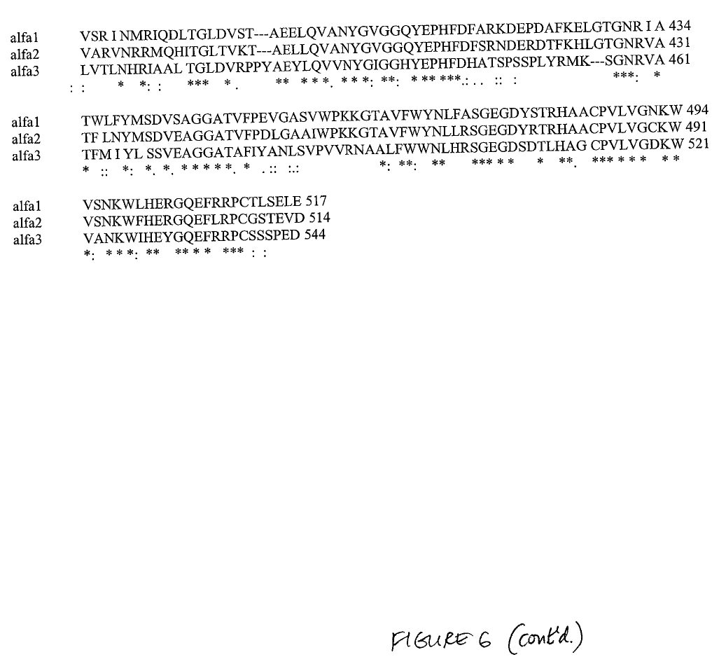

Similarly, fragments of an alpha(III) subunit polypeptide include any polypeptide that is less than the full-length sequence, for example the full-length sequence as depicted in SEQ ID NO:2. Thus, fragments useful in the practice of the present invention can be anywhere from approximately 5 amino acids in length to 543 amino acids in length (or any integer value therebetween). Preferably, the fragments retain one or more biological activities of a full-length alpha(III) isoform (e.g., the ability to form multimers with other prolyl 4-hydroxylase subunits; catalytic functions, etc). Additionally, it is preferred that the fragments comprise at least 10 contiguous amino acid residues of the sequence shown in SEQ ID NO:2. The size of the fragment and region from which it is derived can be determined by one of skill in the art, for example by selecting a fragment based for on homology (or lack thereof) to known sequences as shown in FIG. 6. Once selected, fragments may be constructed by method known in the art including, but not limited to, cleavage of larger fragments and chemical synthesis.

In another embodiment of the invention, one of the polypeptides of the present inventions may be ligated to a heterologous sequence to encode a fusion protein. For example, a fusion protein may be engineered to contain a cleavage site located between an alpha(III) subunit sequence and the heterologous protein sequence, so that the alpha(III) subunit may be cleaved away from the heterologous moiety.

The polypeptides described herein can be isolated or can be produced using chemical methods to synthesize the desired alpha(III) subunit amino acid sequence at least in part. For example, peptides can be synthesized by solid phase techniques, cleaved from the resin, and purified by preparative high performance liquid chromatography. The composition of the synthetic peptides may be confirmed, for example, by amino acid analysis or sequencing.

Expression of the Prolyl 4-Hydroxylase Isoforms

In order to express the alpha subunit isoforms of the invention, the polynucleotide encoding, for example, an alpha(III) subunit or a functional equivalent thereof is inserted into an appropriate expression vector, i.e., a vector which contains the necessary elements for the transcription and translation of the inserted coding sequence, or in the case of an RNA viral vector, the necessary elements for replication and translation.

In a preferred embodiment, the alpha(III) subunits of prolyl 4-hydroxylase of the present invention are co-expressed by the host cell with a subunit of prolyl 4-hydroxylase, so that an active α₂β₂ tetramer is produced. In another aspect, the alpha(III) subunits of prolyl 4-hydroxylase of the present invention are co-expressed by the host cell with a β subunit of prolyl 4-hydroxylase and/or a collagen coding sequence, as described generally in PCT Application No. PCT/US92/09061 (WO 93/07889), such that an α₂β₂ prolyl 4-hydroxylase tetramer is formed and this enzyme catalyzes the formation of 4-hydroxyproline in the expressed collagen.

Methods which are well known to those skilled in the art can be used to construct expression vectors containing the coding sequence for an alpha(III) subunit of the present invention and appropriate transcriptional/translational control signals. These methods include in vitro recombinant DNA techniques, synthetic techniques and in vivo recombination/genetic recombination. See, for example, the techniques described in Maniatis et al., Molecular Cloning A Laboratory Manual, Cold Spring Harbor Laboratory, N.Y. (1989) and Ausubel et al., Current Protocols in Molecular Biology, Greene Publishing Associates and Wiley Interscience, N.Y. (1989).

A variety of host expression vector systems may be utilized. These include, but are not limited to, microorganisms such as bacteria transformed with recombinant bacteriophage DNA, plasmid DNA, or cosmid DNA expression vectors; yeast or filamentous fungi transformed with recombinant yeast or fungi expression vectors; insect cell systems infected with recombinant virus expression vectors (e.g., baculovirus); plant cell systems infected with recombinant virus expression vectors (e.g., cauliflower mosaic virus, CaMV; tobacco mosaic virus, TMV) or transformed with recombinant plasmid expression vectors (e.g., Ti plasmid); or animal cell systems. The expression elements of these systems vary in their strength and specificities. Depending on the host/vector system utilized, any of a number of suitable transcription and translation elements, including constitutive and inducible promoters, may be used in the expression vector containing a polynucleotide of the present invention.

Bacterial Expression Systems

In bacterial systems, a number of expression vectors may be advantageously selected depending upon the use intended for the polypeptide expressed. For example, when large quantities of the isoenzyme subunits of the present invention are to be produced, such as, for example, for use in methods of producing recombinant collagens and gelatins, vectors which direct the expression of high levels of fusion protein products that are readily purified may be desirable. Such vectors include, but are not limited to, the E. coli expression vector pUR278 (Ruther et al. (1983) EMBO J. 2:1791), in which the polypeptide coding sequence may be ligated into the vector in frame with the lac Z coding region so that a hybrid AS-lac Z protein is produced; pIN vectors (Inouye et al. (1985) Nucleic Acids Res. 13:3101-3109; Van Heeke et al. (1989) J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned polypeptide of interest can be released from the GST moiety.

Yeast Expression Systems

A preferred expression system is a yeast expression system. In yeast, a number of vectors containing constitutive or inducible promoters may be used. (See, e.g., Current Protocols in Molecular Biology, Vol. 2, Ed. Ausubel et al., Greene Publish. Assoc. & Wiley Interscience, Ch. 13 (1988); Grant et al., Expression and Secretion Vectors for Yeast, in Methods in Enzymology, Ed. Wu & Grossman, Acad. Press, N.Y. 153:516-544 (1987); Glover, DNA Cloning, Vol. II, IRL Press, Wash., D.C., Ch. 3 (1986); Bitter, Heterologous Gene Expression in Yeast, in Methods in Enzymology, Eds. Berger & Kimmel, Acad. Press, N.Y. 152:673-684 (1987); and The Molecular Biology of the Yeast Saccharomyces, Eds. Strathem et al., Cold Spring Harbor Press, Vols. I and II (1982).)

Alpha(III) subunit proteins of the present invention can be expressed using host cells, for example, from the yeast Saccharomyces cerevisiae. This particular yeast can be used with any of a large number of expression vectors. One of the most commonly employed expression vectors is the multi-copy 2 μ plasmid that contains sequences for propagation both in yeast and E. coli, a yeast promoter and terminator for efficient transmission of the foreign gene. Typical examples of such vectors based on 2 μ plasmids are pWYG4 that has the 2 μ ORI-STB elements, the GALI promoter, and the 2 μ D gene terminator. In this vector an Ncol cloning site is used insert the gene for either the α or β subunit of prolyl 4-hydroxylase, and provide the ATG start codon for the α subunit. As another example, the expression vector can be pWYG7L that has intact 2α ORI, STB, REP1 and REP2, the GAL7 promoter, and uses the FLP terminator. In this vector, the gene for the alpha subunit of prolyl 4-hydroxylase is inserted in the polylinker with its 5′ ends at a BamHI or Ncol site. The vector containing the prolyl 4-hydroxylase gene is transformed into S. cerevisiae either after removal of the cell wall to produce spheroplasts that take up DNA on treatment with calcium and polyethylene glycol or by treatment of intact cells with lithium ions. Alternatively, DNA can be introduced by electroporation.

Transformants can be selected by using host yeast cells that are auxotrophic for leucine, tryptophane, uracil or histidine together with selectable marker genes such as LEU2, TRO1, URA3, HIS3 or LEU2-D. Expression of the alpha(III) subunit prolyl 4-hydroxylase genes driven by the galactose promoters can be induced by growing the culture on a non-repressing, non-inducing sugar so that very rapid induction follows addition of galactose; by growing the culture in glucose medium and then removing the glucose by centrifugation and washing the cells before resuspension in galactose medium; and by growing the cells in medium containing both glucose and galactose so that the glucose is preferentially metabolized before galactose-induction can occur. Further manipulations of the transformed cells are performed as described above to incorporate genes for both the βsubunits of prolyl 4-hydroxylase and desired alpha(III) subunit genes into the cells to achieve expression of an active prolyl 4-hydroxylase αβ tetramer that can be used, for example, in methods of recombinant collagen production to adequately hydroxylate co-expressed collagen genes to fold into a stable triple helical conformation and therefore accompanied by the requisite folding associated with normal biological function. (See, e.g., U.S. Pat. Nos. 5,405,757 and 5,593,859, incorporated herein by reference in their entirety.)

A particularly preferred system useful for cloning and expression of the alpha(III) subunit polypeptides of the present invention uses host cells from the yeast Pichia. Species of non-Saccharomyces yeast such as Pichia pastoris appear to have special advantages in producing high yields of recombinant protein in scaled up procedures. Additionally, a Pichia expression kit is available from Invitrogen Corporation (San Diego, Calif.).

There are a number of methanol responsive genes in methylotrophic yeasts such as Pichia pastoris, the expression of each being controlled by methanol responsive regulatory regions (also referred to as promoters). Any of such methanol responsive promoters are suitable for use in the practice of the present invention. Examples of specific regulatory regions include the promoter for the primary alcohol oxidase gene from Pichia pastoris AOX1, the promoter for the secondary alcohol oxidase gene from P. pastoris AXO2, the promoter for the dihydroxyacetone synthase gene from P. pastoris (DAS), the promoter for the P40 gene from P. pastoris, the promoter for the catalase gene from P. pastoris, and the like.

Another particularly preferred yeast expression system makes use of the methylotrophic yeast Hansenula polymorpha. Growth on methanol results in the induction of key enzymes of the methanol metabolism, namely MOX (methanol oxidase), DAS (dihydroxyacetone synthase) and FMHD (formate dehydrogenase). These enzymes can constitute up to 30-40% of the total cell protein. The genes encoding MOX, DAS, and FMDH production are controlled by very strong promoters which are induced by growth on methanol and repressed by growth on glucose. Any or all three of these promoters may be used to obtain high level expression of heterologous genes in H. polymorpha. The gene encoding an alpha(III) subunit prolyl 4-hydroxylase of the present invention is cloned into an expression vector under the control of an inducible H. polymorpha promoter. If secretion of the product is desired, a polynucleotide encoding a signal sequence for secretion in yeast, is fused in frame with the coding sequence for the present polypeptides. The expression vector preferably contains an auxotrophic marker gene, such as URA3 or LEU2, which may be used to complement the deficiency of an auxotrophic host.

The expression vector is then used to transform H. polymorpha host cells using techniques known to those of skill in the art. An interesting and useful feature of H. polymorpha transformation is the spontaneous integration of up to 100 copies of the expression vector into the genome. In most cases, the integrated DNA forms multimers exhibiting a head-to-tail arrangement. The integrated foreign DNA has been shown to be mitotically stable in several recombinant strains, even under non-selective conditions. This high copy integration further adds to the high productivity potential of the system.

Fungal Expression Systems

Filamentous fungi may also be used to produce the polypeptides of the present invention. Vectors for expressing and/or secreting recombinant proteins in filamentous fungi are well known, and one of skill in the art could use these vectors to express the present alpha(III) subunits or functional equivalents thereof.

Plant Expression Systems

The polypeptides of the present invention may be produced using a plant expression system. Expression systems suitable for use is expressing and using the polypeptides of the present invention involve a number of methods that may be used in accordance with the present invention, including in vitro and in vivo recombinant DNA techniques, and any other synthetic or natural recombination. (See, e.g., Transgenic Plants: A Production System for Industrial and Pharmaceutical Proteins, Owen and Pen eds., John Wiliey & Sons, 1996; Transgenic Plants, Galun and Breiman eds, Imperial College Press, 1997; Applied Plant Biotechnology, Chopra, Malik, and Bhat eds., Science Publishers, Inc., 1999.) The expression of sequences encoding the polypeptides of the present invention may be driven by any of a number of promoters. For example, viral promoters such as the 35S RNA and 19S RNA promoters of CaMV (Brisson et al., Nature 310:511-514 (1984), or the coat protein promoter of TMV (Takamatsu et al., EMBO J. 6:307-311 (1987)) may be used; alternatively, plant promoters such as the small subunit of RUBISCO (Coruzzi et al., EMBO J. 3:1671-1680 (1984); Broglie et al., Science 224:838-843 (1984); or heat shock promoters, e.g., soybean hsp17.5-E or hsp17.3-B (Gurley et al., Mol. Cell. Biol. 6:559-565 (1986) may be used. These constructs can be introduced into plant cells using Ti plasmids, Ri plasmids, plant virus vectors, direct DNA transformation, microinjection, electroporation, etc. Reviews of such techniques are available in the art. (See, e.g., Weissbach & Weissbach, Methods for Plant Molecular Biology, Academic Press, NY, Section VIII, pp. 421-463 (1988); and Grierson & Corey, Plant Molecular Biology, 2d Ed., Blackie, London, Ch. 7-9 (1988).) In a preferred embodiment of the present invention, the present polynucleotides are co-expressed with nucleic acid sequences encoding a β subunit of prolyl 4-hydroxylase under conditions suitable for expression of an active αβ tetramer to produce a biologically active prolyl 4-hydroxylase enzyme.

Various expression vectors may be used to express the alpha(III) subunits using transgenic plants. For example, a typical expression vector contains: prokaryotic DNA elements coding for a bacterial replication origin and an antibiotic resistance gene to provide for the growth and selection of the expression vector in the bacterial host; a cloning site for insertion of an exogenous nucleotide sequence; eukaryotic DNA elements that control initiation of transcription of the exogenous gene, such as a promoter; and DNA elements that control the processing of transcripts, such as a transcription termination/polyadenylation sequence. It also can contain such sequences as are needed for the eventual integration of the vector into the chromosome. In addition, a gene that codes for a selection marker which is functionally linked to promoters that control transcription initiation may also be within the expression vector. Plant expression vectors and reporter genes are generally known in the art. (See, e.g., Gruber et al., 1993, in Methods of Plant Molecular Biology and Biotechnology, CRC Press.)

Typically, the expression vector comprises a nucleic acid construct generated, for example, recombinantly or synthetically, and comprising a promoter that functions in a plant cell, wherein such promoter is operably linked to a nucleic acid sequence encoding an alpha(III) subunit of the present invention.

To produce a desired level of protein expression in plants, the expression of the present polypeptides may be under the direction of a plant promoter. Promoters suitable for use in accordance with the present invention are described in the art. (See, e.g., PCT Publication No. WO 91/19806, incorporated by reference herein in its entirety.) Examples of promoters that may be used in accordance with the present invention include non-constitutive promoters or constitutive promoters. Examples of these types of promoter include the promoter for the small subunit of ribulose-1,5-bis-phosphate carboxylase; promoters from tumor-inducing plasmids of Agrobacterium tumefaciens, such as the RUBISCO nopaline synthase (NOS) and octopine synthase promoters; bacterial T-DNA promoters such as mas and ocs promoters; or viral promoters such as the cauliflower mosaic virus (CaMV) 19S and 35S promoters or the figwort mosaic virus 35S promoter.

In one preferred embodiment, the polynucleotide sequence is under the control of the cauliflower mosaic virus (CaMV) 35S promoter. The double-stranded caulimorvirus family has provided the single most important promoter expression for transgene expression in plants, in particular, the (CaMV) 35S promoter. (See, e.g., Kay et al., 1987, Science 236:1299.) Additional promoters from this family such as the figwort mosaic virus promoter, the Commelina yellow mottle virus promoter, and the rice tungro bacilliform virus promoter have been described in the art, and may also be used in accordance with the present invention. (See, e.g., Sanger et al., 1990, Plant Mol. Biol. 14:433-443; Medberry et al., 1992, Plant Cell 4:195-192; Yin and Beachy, 1995, Plant J. 7:969-980.)

The promoters used in the DNA constructs of the present invention may be modified, if desired, to affect their control characteristics. For example, the CaMV35S promoter may be ligated to the portion of the RUBISCO gene that represses the expression of RUBISCO in the absence of light, to create a promoter which is active in leaves, but not in roots. The resulting chimeric promoter may be used as described herein. Constitutive plant promoters having general expression properties known in the art may be used to express the polypeptides of the present invention. These promoters are abundantly expressed in most plant tissues and include, for example, the actin promoter and the ubiquitin promoter. (See, e.g., McElroy et al., 1990, Plant Cell 2:163-171; Christensen et al., 1992, Plant Mol. Biol. 18:675-689.)

Alternatively, the present enzyme subunits may be expressed in a specific tissue, cell type, or under more precise environmental conditions or developmental control. Promoters directing expression in these instances are known as inducible promoters. In the case where a tissue-specific promoter is used, protein expression is particularly high in the tissue from which extraction of the protein is desired. Depending on the desired tissue, expression may be targeted to the endosperm, aleurone layer, embryo (or its parts as scutellum and cotyledons), pericarp, stem, leaves tubers, roots, etc. Examples of known tissue-specific promoters include the tuber-directed class I patatin promoter, the promoters associated with potato tuber ADPGPP genes, the soybean promoter of β-conglycinin (7S protein) which drives seed-directed transcription, and seed-directed promoters from the zein genes of maize endosperm. (See, e.g., Bevan et al., 1986, Nucleic Acids Res. 14: 4625-38; Muller et al., 1990, Mol. Gen. Genet. 224: 136-46; Bray, 1987, Planta 172: 364-370; Pedersen et al., 1982, Cell 29: 1015-26.)

In one embodiment, the prolyl 4-hydroxylase subunits of the present invention are produced from seed by way of seed-based production techniques using, for example, canola, corn, soybeans, rice and barley seed, and the enzyme product is recovered during seed germination. (See, e.g., PCT Publication Numbers WO 9940210; WO 9916890; WO 9907206; U.S. Pat. No. 5,866,121; and U.S. Pat. No. 5,792,933; and all references cited therein.)

Promoters that may be used to direct the expression of the present polypeptides may be both heterologous or non-hetrologous. These promoters can also be used to drive expression of antisense nucleic acids to reduce, increase, or alter concentration and composition of the various isoforms of prolyl 4-hydroxylase subunits in a desired tissue.

Other modifications may be made to increase and/or maximize transcription of the alpha(III) subunits or functional equivalents thereof are standard and known to those in the art. For example, the nucleic acid construct comprising a polynucleotide encoding an alpha(III) subunit operably linked to a promoter may further comprise at least one factor that modifies the transcription rate of the alpha(II) subunit, including, but not limited to, peptide export signal sequence, codon usage, introns, polyadneylation, and transcription termination sites. Methods of modifying nucleic acid constructs to increase expression levels in plants are generally known in the art. (See, e.g. Rogers et al., 1985, J. Biol. Chem. 260:3731; Cornejo et al., 1993, Plant Mol Biol 23:567-58.) In engineering a plant system that affects the rate of transcription of collagen and related post-translational enzymes, various factors known in the art, including regulatory sequences such as positively or negatively acting sequences, enhancers and silencers, as well as chromatin structure can affect the rate of transcription in plants. The present invention provides that at least one of these factors may be utilized in engineering plants to express the present polypeptides.

The vectors comprising an enzyme subunit coding sequence and any required post-translational enzymes will typically comprise a marker gene which confers a selectable phenotype on plant cells. Usually, the selectable marker gene will encode antibiotic resistance, with suitable genes including at least one set of genes coding for resistance to the antibiotic spectinomycin, the streptomycin phophotransferase (SPT) gene coding for streptomycin resistance, the neomycin phophotransferase (NPTH) gene encoding kanamycin or geneticin resistance, the hygromycin resistance, genes coding for resistance to herbicides which act to inhibit the action of acetolactate synthase (ALS), in particular, the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phophinothricin or basta (e.g. the bar gene), or other similar genes known in the art. The bar gene encodes resistance to the herbicide basta, the nptII gene encodes resistance to the antibiotics kanamycin and geneticin, and the ALS gene encodes resistance to the herbicide chlorsulfuron.

Typical vectors useful for expression of foreign genes in plants are well known in the art, including, but not limited to, vectors derived from the tumor-inducing (Ti) plasmid of Agrobacterium tumefaciens. These vectors are plant integrating vectors, that upon transformation, integrate a portion of the DNA into the genome of the host plant. (See, e.g., Rogers et al., 1987, Meth. In Enzymol. 153:253-277; Schardl et al., 1987, Gene 61:1-11; Berger et al., Proc. Natl. Acad. Sci. U.S.A. 86:8402-8406.)

As mentioned above, vectors comprising a polynucleotide of the present invention and vectors comprising post-translational enzymes or other polypeptides, such as those encoding the β subunit of prolyl 4-hydroxylase, may be co-introduced into the desired plant. Procedures for transforming plant cells are available in the art, including, direct gene transfer, in vitro protoplast transformation, plant virus-mediated transformation, liposome-mediated transformation, microinjection, electroporation, Agrobacterium mediated transformation, and ballistic particle acceleration. (See, e.g., Paszkowski et al., 1984, EMBOJ. 3:2717-2722; U.S. Pat. No. 4,684,611; European application No. 0 67 553 and U.S. Pat. No. 4,407,956; U.S. Pat. No. 4,536,475; Crossway et al., 1986, Biotechniques 4:320-334; Riggs et al., 1986, Proc. Natl. Acad. Sci USA 83:5602-5606; Hinchee et al., 1988, Biotechnology 6:915-921; U.S. Pat. No. 4,945,050.) Standard methods for the transformation of rice, wheat, corn, sorghum, and barley are described in the art. (See, e.g., Christou et al., Trends in Biotechnology 10: 239 (1992), and Lee et al., Proc. Natl. Acad. Sci. USA 88:6389 (1991).) Wheat can be transformed by techniques similar to those employed for transforming corn or rice. Furthermore, Casas et al., Proc. Nat'l Acad. Sci. USA 90: 11212 (1993), describe a method for transforming sorghum, while Wan et al., Plant Physiol. 104: 37 (1994), teach a method for transforming barley. Suitable methods for corn transformation are provided by Fromm et al., Bio/Technology 8: 833 (1990), and by Gordon-Kamm et al., supra.

Additional methods that may be used to generate plants that produce the enzyme isoform subunits and the active complexes of the present invention, and that can be used in related methods of collagen production using the instant isoforms, have been well-established in the art. (See, e.g., U.S. Pat. No. 5,959,091; U.S. Pat. No. 5,859,347; U.S. Pat. No. 5,763,241; U.S. Pat. No. 5,659,122; U.S. Pat. No. 5,593,874; U.S. Pat. No. 5,495,071; U.S. Pat. No. 5,424,412; U.S. Pat. No. 5,362,865; U.S. Pat. No. 5,229,112; U.S. Pat. No. 5,981,841; U.S. Pat. No. 5,959,179; U.S. Pat. No. 5,932,439; U.S. Pat. No. 5,869,720; U.S. Pat. No. 5,804,425; U.S. Pat. No. 5,763,245; U.S. Pat. No. 5,716,837; U.S. Pat. No. 5,689,052; U.S. Pat. No. 5,633,435; U.S. Pat. No. 5,631,152; U.S. Pat. No. 5,627,061; U.S. Pat. No. 5,602,321; U.S. Pat. No. 5,589,612; U.S. Pat. No. 5,510,253; U.S. Pat. No. 5,503,999; U.S. Pat. No. 5,378,619; U.S. Pat. No. 5,349,124; U.S. Pat. No. 5,304,730; U.S. Pat. No. 5,185,253; U.S. Pat. No. 4,970,168; European Publication No. EPA 00709462; European Publication No. EPA 00578627; European Publication No. EPA 00531273; European Publication No. EPA 00426641; PCT Publication No. WO 99/31248; PCT Publication No. WO 98/58069; PCT Publication No. WO 98/45457; PCT Publication No. WO 98/31812; PCT Publication No. WO 98/08962; PCT Publication No. WO 97/48814; PCT Publication No. WO 97/30582; and PCT Publication No. WO 9717459.)

Insect Expression Systems

An alternative expression system used to express the alpha(III) subunits of the present invention is an insect system. Baculoviruses are efficient expression vectors for the large scale production of various recombinant proteins in insect cells. Baculoviruses can be employed to construct expression vectors containing a collagen coding sequence for the collagens of the invention and the appropriate transcriptional/translational control signals. (See, e.g., Luckow et al., 1989, Virology 170:31-39 and Gruenwald, S. and Heitz, J., 1993, Baculovirus Expression Vector System: Procedures & Methods Manual, Pharmingen, San Diego, Calif.) For example, recombinant production of proteins can be achieved in insect cells by infection of baculovirus vectors containing the present polynucleotides.

Specifically, production of an active prolyl 4-hydroxylase can involve the co-infection of insect cells with two baculoviruses, one encoding an subunit of prolyl 4-hydroxylase, the alpha(III) subunit, and another encoding S subunit of prolyl 4-hydroxylase. This insect cell system allows for these recombinant proteins to be produced in large quantities.

In one such system, Autographa californica nuclear polyhidrosis virus (AcNPV) is used as a vector to express foreign genes. The virus grows in Spodoptera frugiperda cells. Coding sequences may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (see, e.g., Smith et al., J. Virol. 46:584 (1983); Smith, U.S. Pat. No. 4,215,051). Further examples of this expression system may be found in Current Protocols in Molecular Biology, Vol. 2, Ed. Ausubel et al., Greene Publish. Assoc. & Wiley Interscience.

Animal Expression Systems

Transgenic animals may also be used to express the polypeptides of the present invention. In one embodiment, the animal is a mammal, and the system is constructed by operably linking a nucleic acid sequence encoding collagen to a promoter and other required or optional regulatory sequences capable of effecting expression in mammary glands, so that the enzyme product is recovered from the milk of the transgenic mammal. Likewise, required or optional post-translational enzymes may be produced simultaneously in the target cells employing suitable expression systems. Methods of using transgenic animals to recombinantly produce proteins are known in the art. For expression in milk, the promoter of choice is preferably be from one of the abundant milk-specific proteins, such as alpha S1-casein, or b-lactoglobulin. For example, 5′ and 3′ regulatory sequences of alpha S1-casein have been successfully used for the expression of the human lactoferrin cDNA, and similarly, the b-lactoglobin promoter has effected the expression of human antitrypsin gene fragments in sheep milk producing cells. (See, e.g., Wright et al., Biotechnology (1991) 9:830-833.) In transgenic goats, the whey acid promoter has been used for the expression of human tissue plasminogen activator, resulting in the secretion of human tissue plasminogen activator in the milk of the transgenic animals. (See, e.g., Ebert et al., Biotechnology (1991) 9:835-838.) Thus, using procedures well-known by those of the ordinary skill in the art, the gene encoding the desired prolyl 4-hydroxylase subunit can simply be ligated to suitable control sequences which function in the mammary cells of the chosen animal species, and the polypeptides of the present invention can be recovered from the milk of these transgenic animals.

In mammalian host cells, a number of viral based expression systems may be utilized. In cases where an adenovirus is used as an expression vector, coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the encoded polypeptides in infected hosts. (See, e.g., Logan & Shenk, Proc. Natl. Acad. Sci. USA 81:3655-3659 (1984)). Alternatively, the vaccinia 7.5 K promoter may be used. (See, e.g., Mackett et al., Proc. Natl. Acad. Sci. USA 79:7415-7419 (1982); Mackett et al., J. Virol. 49:857-864 (1984); Panicali et al., Proc. Natl. Acad. Sci. USA 79:4927-4931 (1982).

A preferred expression system in mammalian host cells is the Semliki Forest virus. Infection of mammalian host cells, for example, baby hamster kidney (BHK) cells and chinese hamster ovary (CHO) cells can yield very high recombinant expression levels. Semliki Forest virus is a preferred expression system as the virus has a broad host range such that infection of mammalian cell lines will be possible. More specifically, it is expected that the use of the Semliki Forest virus can be used in a wide range of hosts, as the system is not based on chromosomal integration, and therefore will be a quick way of obtaining modifications of the recombinant polypeptides in studies aiming at identifying structure-function relationships and testing the effects of various hybrid molecules, such as mouse/human αβ hybrids. Methods for constructing Semliki Forest virus vectors for expression of exogenous proteins in mammalian host cells are described in, for example, Olkkonen et al., 1994, Methods Cell Biol 43:43-53.

Regulatory Elements and Signaling

Specific initiation signals may also be required for efficient translation of inserted prolyl 4-hydroxylase enzyme coding sequences. These signals include the ATG initiation codon and adjacent sequences. In cases where the entire gene, for example, the alpha(III) subunit gene, including its own initiation codon and adjacent sequences, is inserted into the appropriate expression vector, no additional translational control signals may be needed. However, in cases where only a portion of the coding sequence is inserted, exogenous translational control signals, including the ATG initiation codon, must be provided. Furthermore, the initiation codon must be in phase with the reading frame of the coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See, e.g., Bittner et al., Methods in Enzymol. 153:516-544 (1987).)

In a preferred embodiment, the polypeptides of the present invention are expressed as secreted proteins. When the engineered cells used for expression of the proteins are non-human host cells, it is often advantageous to replace the secretory signal peptide of the protein with an alternative secretory signal peptide which is more efficiently recognized by the host cell's secretory targeting machinery. The appropriate secretory signal sequence is particularly important in obtaining optimal fungal expression of mammalian genes. (See, e.g., Brake et al., Proc. Natl. Acad. Sci. USA 81:4642 (1984).) Other signal sequences for prokaryotic, yeast, fungi, insect or mammalian cells are well known in the art, and one of ordinary skill could easily select a signal sequence appropriate for the host cell of choice, and insert the sequence as appropriate, for example, insert a sequence for proteolytic processing and secretion at the N-terminal of the coding sequence.

The vectors of this invention may autonomously replicate in the host cell, or may integrate into the host chromosome. Suitable vectors with autonomously replicating sequences (“ars”) are well known for a variety of bacteria, yeast, and various viral replications sequences for both prokaryotes and eukaryotes. Vectors may integrate into the host cell genome when they have a DNA sequence that is homologous to a sequence found in the host cell's genomic DNA.

In a preferred embodiment, the expression vectors of the invention additionally encode a selection gene, also termed a selectable marker, that encodes a product necessary for the host cell to grow and survive under certain conditions. Typical selection genes include genes encoding (1) a protein that confers resistance to an antibiotic or other toxin (e.g., tetracycline, ampicillin, neomycin, methotrexate, etc.), and (2) a protein that complements an auxotrophic requirement of the host cell, etc. Other examples of selection genes include: the herpes simplex virus thymidine kinase, hypoxanthine-guanine phosphoribosyltransferase, and adenine phosphoribosyltransferase genes that can be employed in tk⁻, hgprt⁻ or aprt⁻ cells, respectively. Also, antimetabolite resistance can be used as the basis of selection for dhfr, which confers resistance to methotrexate; gpt, which confers resistance to mycophenolic acid (Mulligan et al., Proc. Natl. Acad. Sci. USA 78:2072 (1981)); neo, which confers resistance to the aminoglycoside G-418; and hygro, which confers resistance to hygromycin. Recently, additional selectable genes have been described, namely trpB, which allows cells to utilize indole in place of tryptophan; hisD, which allows cells to utilize histinol in place of histidine; and ODC (ornithine decarboxylase) which confers resistance to the omithine decarboxylase inhibitor, 2-(difluoromethyl)-DL-ornithine, DFMO.

Further regulatory elements necessary for the expression vectors of the invention include sequences for initiating transcription, e.g., promoters and enhancers. Promoters are untranslated sequences located upstream from the start codon of the structural gene that control the transcription of the nucleic acid under its control. Inducible promoters are promoters that alter their level of transcription initiation in response to a change in culture conditions, e.g., the presence or absence of a nutrient. One of skill in the art would know of a large number of promoters that would be recognized in host cells suitable for the present invention. These promoters are operably linked to the DNA encoding an enzyme subunit of the present invention by removing the promoter from its native gene and placing the collagen encoding DNA 3′ of the promoter sequence.

Promoters useful in the present invention include, but are not limited to, the following: (prokaryote) (1) the lactose promoter, the alkaline phosphatase promoter, the tryptophan promoter, and hybrid promoters such as the tac promoter, (yeast) (2) the promoter for 3-phosphoglycerate kinase, other glycolytic enzyme promoters (hexokinase, pyruvate decarboxylase, phophofructosekinase, glucose-6-phosphate isomerase, etc.), the promoter for alcohol dehydrogenase, the metallothionein promoter, the maltose promoter, and the galactose promoter, (eukaryotic) (3) virtually all eukaryotic genes have an AT-rich region located approximately 25 to 30 bases upstream from the site where transcription is initiated, examples of suitable eukaryotic promoters include: promoters from the viruses polyoma, fowlpox, adenovirus, bovine papilloma virus, avian sarcoma virus, cytomegalovirus, retroviruses, SV40, and promoters from the target eukaryote including: the glucoamylase promoter from Aspergillus, the actin promoter or an immunoglobin promoter from a mammal, and native collagen promoters.

Transcription

Transcription of the polypeptide-encoding DNA from the promoter is often increased by inserting an enhancer sequence in the vector. Enhancers are cis-acting elements, usually about from 10 to 300 bp, that act to increase the rate of transcription initiation at a promoter. Many enhancers are known for both eukaryotes and prokaryotes, and one of ordinary skill could select an appropriate enhancer for the host cell of interest.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, WI38, etc. Additionally, host cells may be engineered to express various enzymes to ensure the proper processing of the isoenzymes. For example, sequence encoding an alpha subunit of the present invention may be coexpressed with a sequence encoding a β subunit.

For long-term, high-yield production of recombinant proteins, stable expression is preferred. For example, cell lines which stably express the present polypeptides may be engineered. Rather than using expression vectors which contain viral origins of replication, host cells can be transformed with collagen encoding DNA controlled by appropriate expression control elements (e.g., promoter, enhancer, sequences, transcription terminators, polyadenylation sites, etc.), and a selectable marker. Following the introduction of foreign DNA, engineered cells may be allowed to grow for 1 to 2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a desired polypeptide.

The vectors expressing a nucleic acid sequence encoding an alpha(III) subunit or functional equivalent thereof, with or without vectors expressing a β subunit, may be inserted into host cells along with vectors expressing a collagen to produce a desired collagen, using techniques known to one of skill in the art. For example, host cells are transfected or infected or transformed with the above-described expression vectors, and cultured in nutrient media appropriate for selecting transductants or transformants containing the collagen encoding vector. Cell transfection can be carried out by calcium phosphate precipitation, electroporation, and lipofection techniques. (See, e.g., Sambrook et al., 1989, Molecular Cloning. A Laboratory Manual, Cold Spring Harbor Laboratory Press, 2d Edition; Ohta T., 1996, Nippon Rinsho, 1996, 54(3):757-764; Trotter and Wood, 1996, Mol Biotechnol 6(3):329-334; Mann and King, 1989, J Gen Virol 70:3501-3505; and Hartig et al., 1991, Biotechniques 11(3):310.)

Methods of Diagnosis, Prevention, and Treatment

The present invention provides for methods of diagnosing, preventing, and treating various diseases and disorders based on associations with various expression levels of prolyl 4-hydroxylase isoforms. For example, the alpha(I) subunit is dominantly expressed in cardiac tissues, and could thus serve as an appropriate target for treating, preventing, and diagnosing specific diseases and disorders associated with overexpression or underexpression of this isoform or of targets of the isoform's activity. It would thus be appropriate to treat, prevent, or diagnosis diseases and disorders associated with the overexpression or underexpression of this subunit in specific tissues. Similarly, the alpha(III) subunit appears to have significant levels of expression in certain embryonic tissues, and could thus be an appropriate target for methods of treating, preventing, or diagnosing various developmental diseases and disorders associated with overexpression or underexpression of this isoform or peptides or molecules with which it interacts. The alpha(II) isoform appears to be significantly expressed in cultured chondrocytes, suggesting that various therapeutic methods targeting the activity and or the expression of this enzyme isoform could be developed to treat diseases and disorders associated with overdeposition or underdeposition of the cartilage matrix.

Probes and Primers

As noted above, the invention provides various methods for aiding in the diagnosis of the specific disease and disorders associated with aberrant expression of the alpha(III) subunit of prolyl 4-hydroxylase in a biological sample (e.g., cells, tissues, blood, urine, etc.). In certain embodiments, such methods involve the use of polynucleotide described herein and fragments (e.g. probes and primers) of these polynucleotides.

In assaying for alteration in mRNA levels, nucleic acid contained in the aforementioned samples is first extracted according to standard methods known in the art, for example using lytic enzymes or chemical solutions according to the procedures set forth in Sambrook et al, supra or extracted by nucleic-acid-binding resins following the manufacturer's instructions. The mRNA of the polypeptide of interest contained in the sample is then detected by hybridization (e.g., Northern Blot analysis) and/or amplification (e.g., PCR) procedures according to methods known in the art and in view of the teachings herein.

Nucleic acid molecules having at least 10 nucleotides and exhibiting sequence complementarity or homology to the sequences described herein find utility as hybridization probes and, accordingly, in diagnostics and other applications. It is know that a perfectly-matched probe is not needed for a specific hybridization. Minor changes in probe sequence achieved by substitution, deletion or insertion of a small number of bases do not affect hybridization specificity. Typically, as much as 20% base-pair mismatch (when optimally aligned) can be tolerated. Preferably, a probe useful for detecting the prolyl 4-hydroxylase mRNA is at least about 80% identical to the homologous region of SEQ ID NO:1. These probes can be used in radioassays (e.g.,. Southern or northern blot analysis) to detect, prognose, diagnose or monitor various disease states resulting from aberrant expression (e.g., overexpression or underexpression) of the alpha(III) subunit. The total size of the fragment, as well as the size of the complementary stretches, will depend on the intended use or application of the particular nucleic acid segment.

The polynucleotides (e.g., probes) of the present invention can also be used as primers for the detection of differentially expressed alpha(III) subunit genes in certain tissues or cells, for example using these primers to amplify alpha(III) sequences from a biological sample. For the purpose of this invention, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. A preferred amplification method is PCR. General procedures for PCR are taught in MacPherson, et al. PCR: A PRACTICAL APPROACH (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides.

The polynucleotides also can be attached to a solid support such as a chip for use in high throughput screening assays for the detection and monitoring of alpha(III) subunit-related conditions. Accordingly, this invention also provides polynucleotides comprising SEQ ID NO:1, fragments thereof and complements of SEQ ID NO:1 and fragments thereof, attached to a solid support for use in high throughput screens. For attachment to a solid support, the polynucleotides can be synthesized on the solid surface, for example on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

The expression level of alpha(III) subunit is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See U.S. Pat. Nos. 5,578,832; and 5,631,734. The obtained measurement is directly correlated with gene expression level. Results from the chip assay are typically analyzed using a computer software program. See, for example, EP 717113 A2 and WO 95/20681. The hybridization data is read into the program, which calculates the expression level of the targeted gene(s). This figure is compared against existing data sets of gene expression levels for that cell type.

For example, the database and methods of using the database provides a means to differentiate a cell expressing an alpha(III) polypeptide (or fragment thereof) which is the natural counterpart of the peptides described herein from a cell which does not express the polypeptide or expresses it at a higher or lower level from the cell in question. Expression of polynucleotides encoding the peptide is measured. One cell would serve as a “reference cell” and the cell whose expression of a polynucleotide encoding an alpha(III) subunit is to be measured could be referred to as the “test cell”. As an example, the method can be used to distinguish a normal cell (in this case, the reference cell) from a cell (i.e., the test cell) in which alpha(III) is differentially expressed and in which differential expression may be correlated to one or more disease states. As used herein “differential” or “altered” expression refers to increased expression (overexpression) or decreased expression (underexpression) as compared to the selected control. The differential expression can be measured qualitatively (e.g., by examining intensity of straining or other optical assessments) or quantitatively. It can be used to analyze drug toxicity and efficacy, as well as to selectively look at protein categories which are expected to be affected by a drug or which may be overexpressed as a result of treatment with a drug, such as the various multi-drug resistant genes. Additional utilities include, but are not limited to analysis of the developmental state of a test cell, the influence of viral or bacterial infection, polymorphism within the cell type, and the effect of regulatory genes.

In certain embodiments, it will be advantageous to employ nucleic acid sequences of the present invention in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmental undesirable reagents. In the case of enzyme tags, colorimetric indicator substrates are known which can be employed to provide a means visible to the human eye or spectrophotometrically, to identify specific hybridization with complementary nucleic acid-containing samples.

Antibodies

In one embodiment of the present invention, methods for diagnosis, prevention, and treatment of diseases and disorders associated with increased or decreased expression and or activity of various prolyl 4-hydroxylase enzymes involve the administration of a therapeutically effective amount of an antibody which specifically reacts with a particular prolyl 4-hydroxylase isoenzyme.

Expression of novel transcript can also be determined by assaying for the presence of the protein product. Determining the protein level involves (a) providing a biological sample suspected of containing polypeptides; and (b) measuring the amount of any immunospecific binding that occurs between an antibody reactive to the protein product and detecting the presence of any antibody: protein complex formed. The presence of a complex indicates that the protein product was present in the sample and therefore, the sample contained an alpha(III) subunit.

Antibodies may be generated using methods well known in the art. Such antibodies may include, but are not limited to, polyclonal, monoclonal, chimeric, single chain antibodies, as well as Fab fragments, including F(ab′)₂ and Fᵥ fragments. Fragments can be produced, for example, by a Fab expression library. Neutralizing antibodies, i.e., those which inhibit dimer formation, are especially preferred for therapeutic use.

A target polypeptide, such as, for example, the alpha(III) subunit of human prolyl 4-hydroxylase, or an agent that modulates the activity and or expression of the alpha(III) subunit, can be evaluated to determine regions of high immunogenicity. Methods of analysis and epitope selection are well-known in the art. See, e.g., Ausubel, et al., eds., 1988, Current Protocols in Molecular Biology. Analysis and selection can also be accomplished, for example, by various software packages, such as LASERGENE NAVIGATOR software. (DNASTAR; Madison Wis.) The peptides or fragments used to induce antibodies should be antigenic, but are not necessarily biologically active. Preferably, an antigenic fragment or peptide is at least 5 amino acids in length, more preferably, at least 10 amino acids in length, and most preferably, at least 15 amino acids in length. It is preferable that the antibody-inducing fragment or peptide is identical to at least a portion of the amino acid sequence of the target polypeptide, e.g., CTGF. A peptide or fragment that mimics at least a portion of the sequence of the naturally occurring target polypeptide can also be fused with another protein, e.g., keyhole limpet hemocyanin (KLH), and antibodies can be produced against the chimeric molecule.

Methods for the production of antibodies are well-known in the art. For example, various hosts, including goats, rabbits, rats, mice, humans, and others, may be immunized by injection with the target polypeptide or any immunogenic fragment or peptide thereof. Depending on the host species, various adjuvants may be used to increase immunological response. Such adjuvants include, but are not limited to, Freund's adjuvant, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

Monoclonal and polycolonal antibodies may be prepared using any technique which provides for the production of antibody molecules by continuous cell lines in culture. Techniques for in vivo and in vitro production are well-known in the art. See, e.g., Pound, J. D., 1998, Immunochemical Protocols, Humana Press, Totowa N.J.; Harlow, E. and D. Lane, 1988, Antibodies, A Laboratory Manual, Cold Spring Harbor Laboratory, New York. The production of chimeric antibodies is also well-known, as is the production of single-chain antibodies. See, e.g., Morrison, S. L. et al., 1984, Proc. Natl. Acad. Sci. 81:6851-6855; Neuberger, M. S. et al., 1984, Nature 312:604-608; Takeda, S. et al., 1985 Nature 314:452-454. Antibodies with related specificity, but of distinct idiotypic composition, may be generated, for example, by chain shuffling from random combinatorial immunoglobin libraries. See, e.g., Burton D. R., 1991, Proc. Natl. Acad. Sci. 88:11120-11123.

Antibodies may also be produced by inducing in vivo production in the lymphocyte population or by screening immunoglobulin libraries or panels of highly specific binding reagents. (See, e.g., Orlandi, R. et al., 1989, Proc. Natl. Acad. Sci. 86:3833-3837; Winter, G. and C. Milstein, 1991, Nature 349:293-299.) Antibody fragments which contain specific binding sites for the target polypeptide may also be generated. Such antibody fragments include, but are not limited to, F(ab′)₂ fragments, which can be produced by pepsin digestion of the antibody molecule, and Fab fragments, which can be generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D., et al., 1989 Science 254:1275-1281.)

Antibodies can be tested for anti-target polypeptide activity using a variety of methods well-known in the art. Various techniques may be used for screening to identify antibodies having the desired specificity, including various immunoassays, such as enzyme-linked immunosorbent assays (ELISAs), including direct and ligand-capture ELISAs, radioimmunoassays (RIAs), immunoblotting, and fluorescent activated cell sorting (FACS). Numerous protocols for competitive binding or immunoradiometric assays, using either polyclonal or monoclonal antibodies with established specificities, are well known in the art. Such immunoassays typically involve the measurement of complex formation between the target polypeptide and a specific antibody. A two-site, monoclonal-based immunoassay utilizing monoclonal antibodies reactive to two non-interfering epitopes on the target polypeptide is preferred, but other assays, such as a competitive binding assay, may also be employed. (See, e.g., Maddox, D. E., et al, 1983, J Exp Med 158:1211.)

Antibodies as described above could also be used to identify α or β prolyl 4-hydroxylase or fragments thereof in tissue, e.g., biopsies from specific tissues, etc., or other biological samples. The amount of a particular polypeptide present could be determined, for example, by quantitative image analysis. Alternatively, the mRNA of the target polypeptide could be determined, such as by reverse transcriptase polymerase chain reaction (PCR) using a biological sample. In particular, in this method, mRNA from, for example, a tissue sample in total, or that specific for the target polypeptide or fragments thereof, could be transcribed to DNA and then amplified through PCR using specific primer sequences. Quantitation of mRNA could be determined, for example, by a competition reaction using equal volumes of the patient sample run against a series of decreasing known concentrations, e.g., of a mimic or mutant cDNA fragment.

The present invention contemplates the use of antibodies specifically reactive with a target polypeptide, i.e., a prolyl 4-hydroxylase isoform, or fragments thereof, which neutralize the biological activity the of the prolyl 4-hydroxylase isoform. The antibody administered in the method can be the intact antibody or antigen binding fragments thereof, such as Fab, F(ab′)₂, and Fv fragments, which are capable of binding the epitopic determinant. The antibodies used in the method can be polyclonal or, more preferably, monoclonal antibodies. Monoclonal antibodies with different epitopic specificities are made from antigen containing fragments of the protein by methods well known in the art. (See, e.g., Kohler et al., Nature 256:494; Ausubel, et al., supra.)

In the present invention, therapeutic applications include those using “human” or “humanized” antibodies directed to a specific prolyl 4-hydroxylase isoform or fragments thereof. Humanized antibodies are antibodies, or antibody fragments, that have the same binding specificity as a parent antibody, (i.e., typically of mouse origin) and increased human characteristics. Humanized antibodies may be obtained, for example, by chain shuffling or by using phage display technology. For example, a polypeptide comprising a heavy or light chain variable domain of a non-human antibody specific for a prolyl 4-hydroxylase isoform is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method of the present invention are disclosed in, for example, U.S. Pat. Nos. 5,565,332; 5,585,089; 5,694,761; and 5,693,762. Furthermore, techniques described for the production of human antibodies in transgenic mice are described in, for example, U.S. Pat. Nos. 5,545,806 and 5,569,825.

Antisense

Antisense technology relies on the modulation of expression of a target protein through the specific binding of an antisense sequence to a target sequence encoding the target protein or directing its expression. See, e.g., Agrawal, S., ed., 1996, Antisense Therapeutics, Humana Press Inc., Totawa N.J.; Alama, A. et al., 1997, Pharmacol. Res. 36(3):171-178; Crooke, S. T.,1997, Adv. Pharmacol. 40:1-49; and Lavrosky, Y. et al.,1997, Biochem. Mol. Med. 62(1):11-22. Antisense sequences are nucleic acid sequences capable of specifically hybridizing to at least a portion of a target sequence. Antisense sequences can bind to cellular mRNA or genomic DNA, blocking translation or transcription and thus interfering with expression of a targeted protein product. Antisense sequences can be any nucleic acid material, including DNA, RNA, or any nucleic acid mimics or analogs. See, e.g., Rossi, J. J. et al., 1991 Antisense Res. Dev. 1(3):285-288; Pardridge, W. M. et al., 1995, Proc. Nat. Acad. Sci. 92(12):5592-5596; Nielsen, P. E. and G. Haaima, 1997, Chem. Soc. Rev. 96:73-78; and Lee, R. et al., 1998, Biochemistry 37(3):900-1010.). Delivery of antisense sequences can be accomplished in a variety of ways, such as through intracellular delivery using an expression vector. Site-specific delivery of exogenous genes is also contemplated, such as techniques in which cells are first transfected in culture and stable transfectants are subsequently delivered to the target site.

Antisense oligonucleotides of about 15 to 25 nucleic acid bases are typically preferred as such are easily synthesized and are capable of producing the desired inhibitory effect. Molecular analogs of antisense oligonucleotide may also be used for this purpose and can have added advantages such as stability, distribution, or limited toxicity advantageous in a pharmaceutical product. In addition, chemically reactive groups, such ass iron-linked ethylenediamine-tetraacetic acid (EDTA-Fe), can be attached to antisense oligonucleotides, causing cleavage of the RNA at the site of hybridization. These and other uses of antisense methods to inhibit the in vitro translation of genes are well known in the art. See, e.g., Marcus-Sakura, 1988, Anal. Biochem 172:289.

Delivery of antisense therapies and the like can be achieved intracellularly through using a recombinant expression vector such as a chimeric virus or a colloidal dispersion system which, upon transcription, produces a sequence complementary to at least a portion of the cellular sequence encoding the target protein. See, e.g., Slater, J. E. et al., 1998, J. Allergy Cli. Immunol. 102(3):469-475. Delivery of antisense sequences can also be achieved through various viral vectors, including retrovirus and adeno-associated virus vectors. See, e.g., Miller, A. D., 1990, Blood 76:271; and Uckert, W. and W. Walther,1994, Pharacol. Ther. 63(3):323-347. Vectors which can be utilized for antisense gene therapy as taught herein include, but are not limited to, adenoviruses, herpes viruses, vaccinia, or, preferably, RNA viruses such as retroviruses.

Retroviral vectors are preferably derivatives of murine or avian retrovirus. Retroviral vectors can be made target-specific by inserting, for example, a polynucleotide encoding a protein or proteins such that the desired ligand is expressed on the surface of the viral vector. Such ligand may be a glycolipid carbohydrate or protein in nature. Preferred targeting may also be accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the antisense polynucleotide.

Recombinant retroviruses are typically replication defective, and can require assistance in order to produce infectious vector particles. This assistance can be provided by, for example, using helper cell lines that contain plasmids encoding all-of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal may be used. These cell lines produce empty virions, since no genome is packaged. If a retroviral vector is introduced into such cells in which the packaging signal is intact, but the structural genes are replaced by other genes of interest, the vector can be packaged and vector virion produced.

Other gene delivery mechanisms that can be used for delivery of antisense sequences to target cells include colloidal dispersion and liposome-derived systems, artificial viral envelopes, and other systems available to one of skill in the art. See, e.g., Rossi, J. J., 1995, Br. Med. Bull. 51(1):217-225; Morris, M. C. et al., 1997, Nucl. Acids Res. 25(14):2730-2736; and Boado, R. J. et al., 1998, J. Pharm. Sci. 87(11):1308-1315. For example, delivery systems can make use of macromolecule complexes, nanocapsules, microspheres, beads, and lipid-based systems including oil-in-water emulsions, micelles, mixed micelles, and liposomes.

In one embodiment, a method of the present invention administers a therapeutically effective amount of an antisense oligonucleotide having a sequence capable of binding specifically with any sequences of an mRNA molecule which encodes a specific prolyl 4-hydroxylase alpha subunit, so as to prevent translation of prolyl 4-hydroxylase alpha subunit mRNA.

Agonists/Antagonists

The present invention further provides a methods for identifying compounds that demonstrate antagonistic or agonistic activity towards specific isoforms of prolyl 4-hydroxylase. in which small molecules are used to inhibit the activity of prolyl 4-hydroxylase. For example, the present invention provides methods of treating and preventing kidney fibrosis utilizing small molecules that modulate, regulate and inhibit prolyl 4-hydroxylase activity.

This invention encompasses methods of identifying small molecules and other agents useful in the present methods for treating, preventing, and diagnosing various diseases and disorders by modulating the expression and activity of the alpha subunits of prolyl 4-hydroxylase of the present invention. These therapeutic compounds can be identified by any of a variety of screening techniques known in the art.

Fragments employed in such screening tests may be free in solution, affixed to a solid support, borne on a cell surface, or located intracellularly. The blocking or reduction of biological activity or the formation of binding complexes between the alpha subunits of prolyl 4-hydroxylase and the agent being tested can be measured by methods available in the art.

Other techniques for drug screening which provide for a high throughput screening of compounds having suitable binding affinity to one of the instant polypeptides or to another target polypeptide useful in modulating, regulating, or inhibiting the expression and/or activity of the instant polypeptides, are known in the art. For example, microarrays carrying test compounds can be prepared, used, and analyzed using methods available in the art. See, e.g., Shalon, D. et al., 1995, PCT Application No. WO95/35505, Baldeschweiler et al., 1995, PCT Application No. WO 95/251116; Brennan, T. M. et al., 1995, U.S. Pat. No. 5,474,796; Heller, M. J. et al., 1997, U.S. Pat. No. 5, 605,662.

Various assays and screening techniques can be used to identify small molecules that modulate expression and activity of the alpha subunits of prolyl 4-hydroxylase, and can also serve to identify antibodies and other compounds that interact with prolyl 4-hydroxylase and can be used as drugs and therapeutics in the present methods. See, e.g., Enna, S. J. et al., eds., 1998, Current Protocols in Pharmacology, John Wiley and Sons. Assays will typically provide for detectable signals associated with the binding of the compound to a protein or cellular target. Binding can be detected by, for example, fluorophpres, enzyme conjugates, and other detectable labels well-known in the art. The results may be qualitative or quantitative.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods which are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

Isolation and Cloning of the Human alpha(III) Subunit Gene.

The GenBank database of human expressed sequence tags was searched with the human prolyl 4-hydroxylase alpha(I) subunit amino acid sequence, and a 581 bp sequence AA116081 coding for amino acids homologous to the C-terminal end of human prolyl 4-hydroxylase alpha(I) subunit was identified.

The AA116081 sequence information was used to design a 3′ RACE (rapid amplification of cDNA ends) primer β3-r1 (5′-GTT AGG AAT GCA GCA CTG TTT TGG TGG-3′, SEQ ID NO:3) and a 5′ RACE primer α3-r2 (5′-GCT GGA GCT GCA GGG TCT GCG GAA-3′, SEQ ID NO:4). 5′ and 3′ RACE analysis was performed using these primers and primer AP1 (a linker specific primer from the Marathon-Ready cDNA kit, Clontech Laboratories Inc., Palo Alto, Calif.), and a total fetal cDNA pool (Marathon-Ready cDNA, Clontech Laboratories Inc., Palo Alto, Calif.) as a template. The 3′RACE analysis resulted in amplification of a PCR product starting from the α3-r1 primer (nt 1462) and containing 591 bp of 3′ noncoding sequence and a polyA signal followed by the AP1 primer.

This 3′ RACE product was cloned into the pUC18 vector using the Sure Clone ligation kit (Amersham Pharmacia), generating pUC18/alpha(III)1462-polyA (orientation . . . BamHI-alpha(III)1462-polyA-EcoRI . . . ). The 5′RACE analysis resulted in amplification of a PCR product starting from the α3-r2 and containing 1367 bp of the coding sequence (nts 257-1623) followed by the AP1 primer. This 5′ RACE product was cloned into pUC 18 vector using the Sure Clone ligation kit, generating pUC18/alpha(III)257-1623 (orientation . . . BamHI-alpha(III)257-1623-EcoRI . . . ).

To generate pUC18/alpha(III)257-polyA, BamHI-EcoRI digested alpha(II)257-1623 (the alpha(III) cDNA has an internal EcoRI site 1598) and EcoRI digested alpha(III)1462-polyA were cloned into BamHI-EcoRI digested pUC18. A nested 5′RACE analysis was performed with the α3-r10 (5′-AAT GGC ATG GTA ATA ATC CCC CAT GCT ATA-3′, SEQ ID NO:5) and AP2 (a linker specific primer from the Marathon-Ready cDNA kit, Clontech Laboratories Inc., Palo Alto, Calif.) primers. The nested 5′RACE analysis resulted in amplification of a PCR product starting from the α3-r10 and containing 420 bp of the coding sequence (nts 184-603) followed by the AP2 primer.

This 5′ nested RACE product was cloned into the pUC18 vector using the Sure Clone ligation kit, generating pUC18/alpha(III)184-603 (orientation . . . BamHI-alpha(III)184-603-EcoRI . . . ). Further nested 5′RACE analyses did not lead to the identification of the first 183 bp of the 5′ end of the cDNA clone.

Simultaneously with the RACE analyses, PCR primers (α3-1 (5′-GTT AGG AAT GCA GCA CTG TTT T-3′, SEQ ID NO:6) and α3-2 (5′-GCT GGA GCT GCA GGG TCT-3′, SEQ ID NO:7) were designed based on the EST clone AA1 16081 sequence and used to obtain a 162-bp product from a total fetal cDNA pool (Marathon-Ready cDNA, Clontech Laboratories Inc., Palo Alto, Calif.). This 162-bp PCR product was used to screen human placenta 5′-stretch plus (Clontech Laboratories Inc., Palo Alto, Calif.) and human endothelial cell line lambda (Stratagene, La Jolla, Calif.) cDNA libraries. Positive clones were not obtained from the placenta library. 11 positive clones were obtained from the endothelial cell line library and they were characterized in detail. None of the 11 cDNA clones contained the 5′ nucleotides 1-183.

An EST clone AA043201 overlapping with nucleotides 184-288 of the alpha(III) subunit cDNA was identified using GenBank. This EST clone was characterized and found to contain part of the missing 5′ end covering the alpha(III) subunit cDNA nucleotides 34-183. Simultaneously, the genomic sequence for the alpha(II) subunit identified using GenBank No. AC006595, and the 5′ nucleotides 1-34 were determined using this clone.

Using this sequence information, the 5′ end covering the nucleotides 1-183 was generated by PCR using primers α3-16 (5′-ATG GGT CCT GGG GCG CGG CTG GC-3′, SEQ ID NO:8) and α3-19 (5′-CCG CGC CTC CTC CCC GCG CAG GTA-3′, SEQ ID NO:9) and human genomic DNA (Boehringer Mannheim) as a template. The PCR product was cloned into the pUC18 vector using the Sure Clone ligation kit generating pUC18-alpha(III)/1-183 (orientation . . . BamHI-alpha(III)/1-183-EcoRI . . . ).

The full-length alpha(III) subunit cDNA was cloned into the pUC 18 vector. First, a PCR fragment covering alpha(III) nucleotides 1-183 was generated using pUC18forward and α3-19 primers, and pUC18-alpha(III)/1-183 as a template. This PCR product was digested with BamHI. A PCR fragment covering alpha(III) nucleotides 184-603 was generated using α3-17 (5′-CTG CGG GAC CTG ACT AGA TTC TAC-3′, SEQ ID NO:10) and pUC18reverse primers, and pUC 18/alpha(III) 184-603 as a template. This PCR product was digested with ApaI (alpha(III) and possessed an internal ApaI 418 site). These two PCR products were coligated into BamHI-ApaI digested pUC18/alpha(III)257-polyA, generating pUC 18-alpha(III)/1-polyA.

### Example 2

Sequencing

The nucleotide sequences for the clones described in Example 1 are determined by the dideoxynucleotide chain-termination method, as described in Sanger, et al., (1977) Proc. Natl. Acad. Sci. (USA) 74:5463-67, with T7 DNA polymerase (Pharmacia). Vector-specific or sequence-specific primers are in an Applied Biosystems DNA synthesizer (Department of Biochemistry, University of Oulu) and used. The DNASIS and PROSIS version 6.00 sequence analysis software (Pharmacia), ANTHEPROT (as disclosed in Deleage, et al. (1988) Comput. Appl. Biosci. 4:351-356), the Wisconsin Genetics Computer Group package version 8 (September 1994), and BOXSHADE (Kay Hofinann, Bioinformatics Group, Institut Suisse de Recherches Experimentales sur le Cancer Lausanne, Switzerland) are used to compile the sequence data.

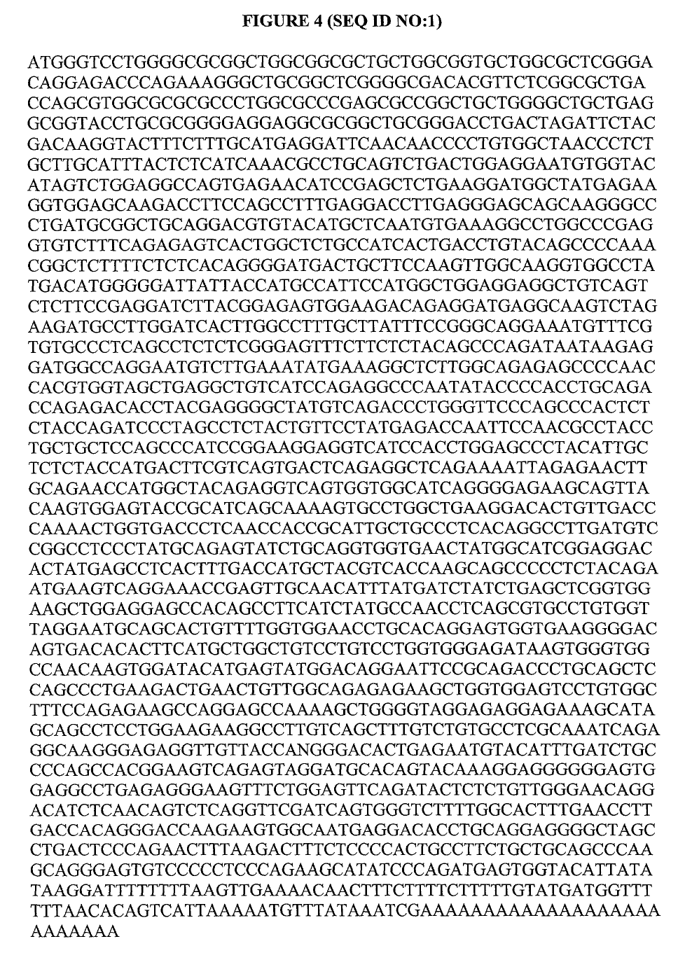

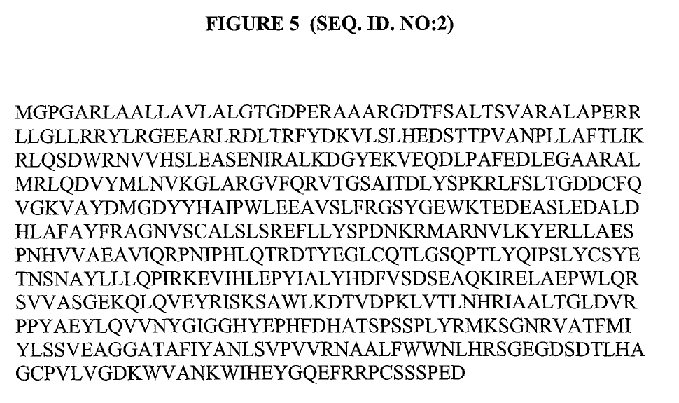

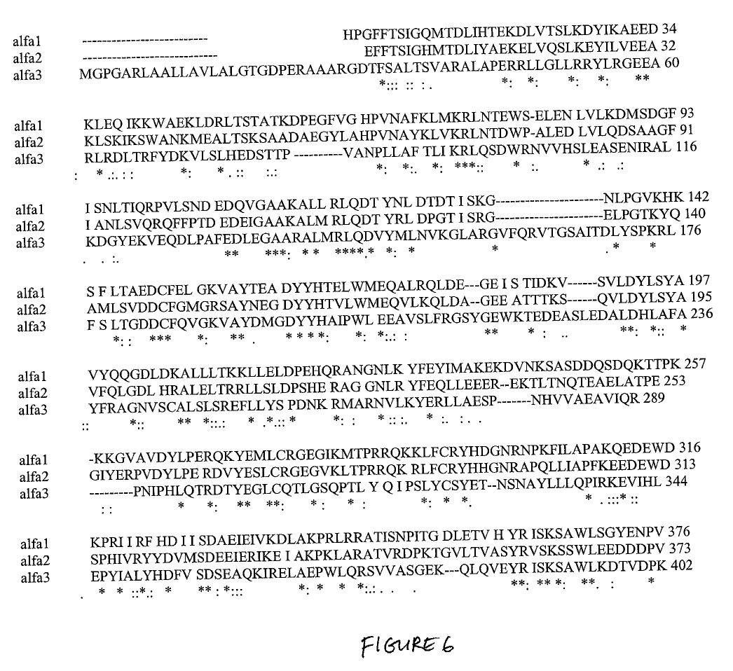

FIG. 4 (SEQ ID NO:1) shows the nucleotide sequence of the coding region of the alpha(III) subunit of prolyl-4-hydroxylase. This sequence encodes a 544 amino acid polypeptide, shown in FIG. 5 (SEQ ID NO:2). Alignment of this polypeptide with other alpha subunits is shown in FIG. 6.

### Example 3

RT-PCR Analysis

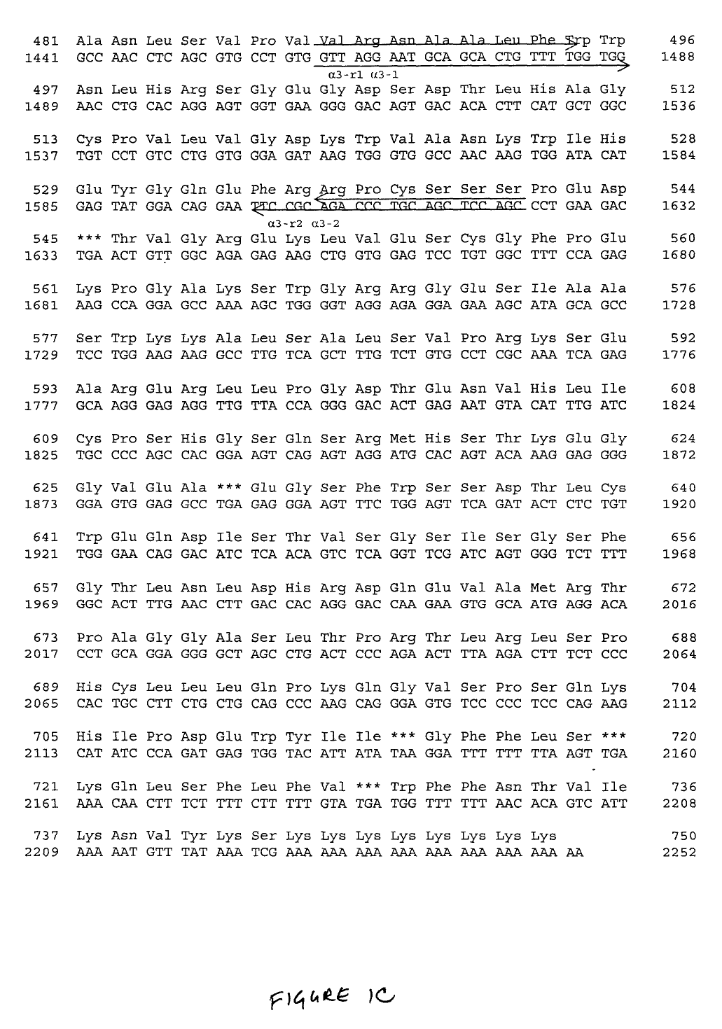

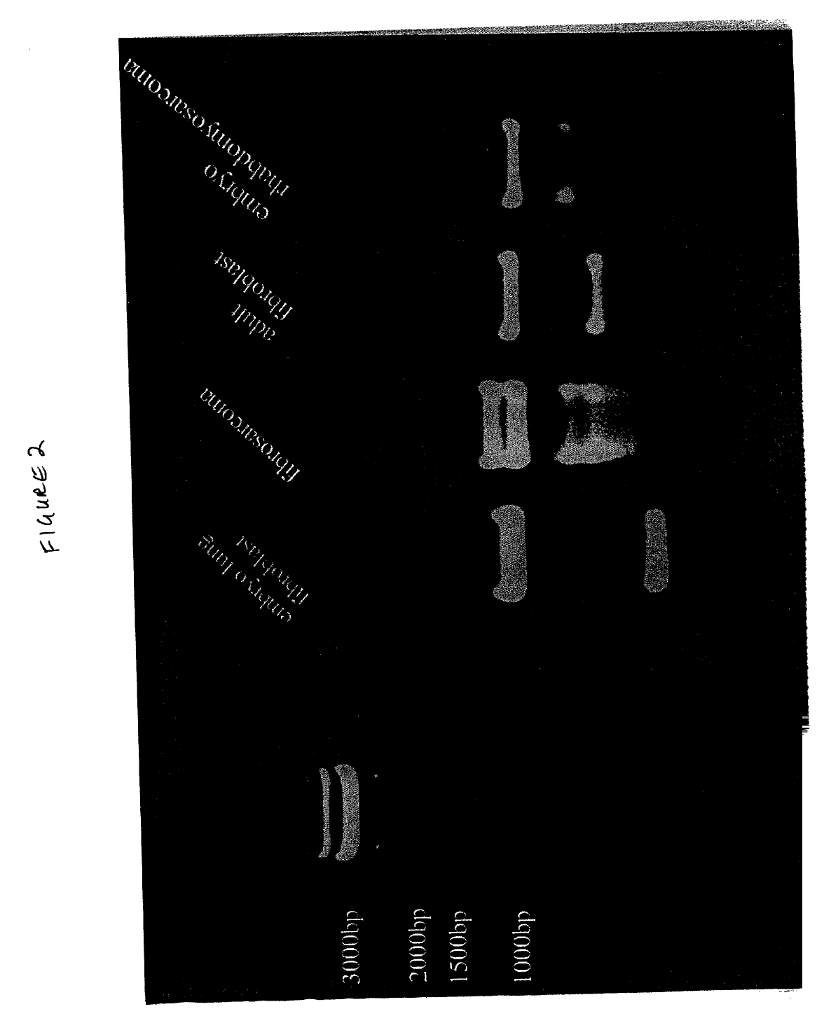

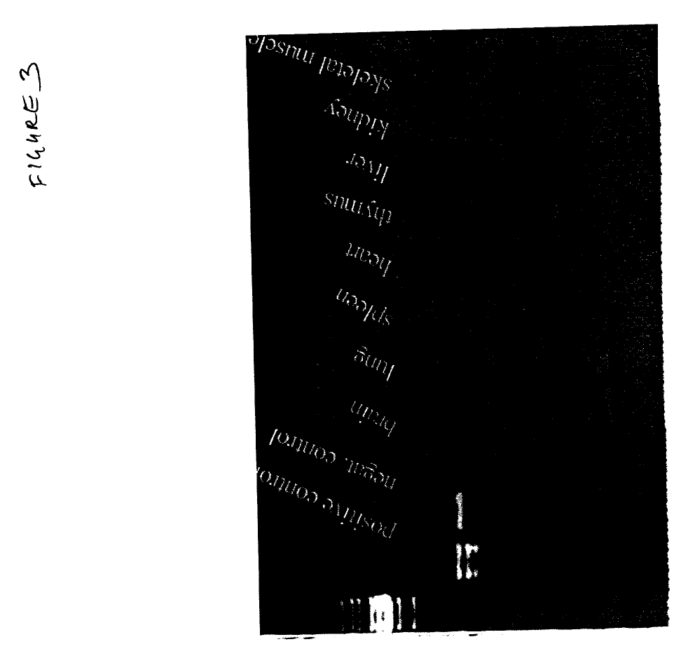

FIGS. 2 represents the results of an RT-PCR assay of the expression of human prolyl 4-hydroxylase alpha(III) subunit mRNA samples isolated from cultured cells. The size of the human alpha(III) subunit is about 1.0 kb. The correct alpha 3 band in this figure is the 1000 bp band (verified by sequencing). FIG. 3 shows the results of another RT-PCR assay, performed using a Human Fetal Multiple Tissue cDNA Panel (Clontech Laboratories Inc., Palo Alto, Calif.).

These results indicate that the alpha(III) subunit shows significant expression levels in certain embryonic tissues, and could thus serve as an appropriate target in various therapeutic methods directed at treatment, prevention, and diagnosis of various diseases and disorders associated with development, etc.

### Example 4

Enzyme Activity Assays

Prolyl 4-hydroxylase activity is assayed, for example, by a method based on the decarboxylation of 2-oxoH¹⁴C-glutarate, as disclosed in Kivirriko and Myllyla (1982) Methods Enzymol. 82:245-304. The kₘ values are determined by varying the concentration of one substrate in the presence of fixed concentrations of the second while the concentrations of the other substrates are kept constant, as set forth in Myllyla, et al., (1977) Eur. J Biochem. 80:349-357.

0.1% Triton X-100 extracts from cell homogenates containing either the mouse-human hybrid or the human alpha1 subunit enzyme are analyzed for prolyl 4-hydroxylase activity with an assay based on the hydroxylation-coupled decarbosylation of 2-oxo[1¹⁴C]glutarate (Kivirikko and Myllyla, supra). To show that the activity of the mouse/human hybrid was prolyl 4-hydroxylase activity, the amount of 4-hydroxyproline in a (Pro-Pro-gly)₁₀ substrate was determined after the reaction. Similar results, including Kₘ values, etc., indicate that the activities of the hybrid and the alpha1 enzymes are similar, and that the activity of the hybrid is thus prolyl 4-hydroxylase activity.

Various modifications of the invention, in addition to those shown and described herein, will become apparent to those skilled in the art from the foregoing description. Such modifications are intended to fall within the scope of the appended claims. It is also to be understood that all base pair sizes given for nucleotides are approximate and are used for purposes of description.

All references cited herein are hereby incorporated by reference in their entirety.

What is claimed is:

1. A substantially purified polypeptide comprising the amino acid sequence of SEQ ID NO:2 or variants or fragments thereof.

2. An isolated and purified polynucleotide encoding the polypeptide of claim 1.

3. An isolated and purified polynucleotide that exhibits at least 80% sequence identity to the polynucleotide of SEQ ID NO:1.

4. An isolated and purified polynucleotide which hybridizes under stringent conditions to the polynucleotide of claim 2.

5. An isolated and purified polynucleotide which hybridizes under stringent conditions to the polynucleotide of claim 3.

6. An isolated and purified polynucleotide which is complementary to the polynucleotide of claim 2.

7. An isolated and purified polynucleotide which is complementary to the polynucleotide of claim 3.

8. An expression vector comprising the polynucleotide of claim 2 or fragments thereof.

9. The expression vector of claim 8 further comprising a nucleotide sequence encoding a β subunit of prolyl 4-hydroxylase.

10. A host cell comprising the polynucleotide of claim 2.

11. A host cell of claim 10, wherein the host cell further comprises one or more polynucleotide sequences encoding a beta subunit of prolyl 4-hydroxylase.

12. The host cell of claim 10, wherein the host cell further comprises one or more nucleotide sequences encoding one or more collagen molecules.

13. The host cell of claim 10, wherein the cell is a eukaryotic cell.

14. The host cell or claim 10, wherein the cell is a prokaryotic cell.

15. The host cell of claim 10, wherein the host cell is selected from the group consisting of insect cells, yeast cells, bacterial cells, plant cells, or mammalian cells.

16. A method for producing a polypeptide, the method comprising:
(a) culturing the host cell of claim 10 under conditions suitable for expression of the polypeptide; and
(b) isolating the polypeptide.

17. A method for producing a prolyl 4-hydroxylase tetramer, the method comprising:
(a) culturing the host cell of claim 11 under conditions suitable for formation of the prolyl 4-hydroxylase tetramer; and
(b) recovering the prolyl 4-hydroxylase tetramer.

18. A method for detecting a polynucleotide in a sample, the method comprising:
(a) hybridizing the polynucleotide of claim 2 to at least one nucleic acid in a sample, thereby forming a hybridization complex; and
(b) detecting the hybridization complex, wherein the presence of the hybridization complex is indicative of the presence of the polynucleotide in the sample.

19. A pharmaceutical composition comprising the polypeptide of claim 1 and a suitable pharmaceutical carrier.

20. A pharmaceutical composition comprising the polynucleotide of claim 2 and a suitable pharmaceutical carrier.

21. A purified antibody which specifically binds to the polypeptide of claim 1.

22. A purified agonist of the polypeptide of claim 1.

23. A purified antagonist of the polypeptide of claim 1.

24. A method for treating or preventing a disorder associated with decreased expression or activity of the alpha(III) subunit of prolyl 4-hydroxylase, the method comprising administering to a subject in need an effective amount of the pharmaceutical composition of claim 19.

25. A method for treating or preventing a disorder associated with decreased expression or activity of the alpha(III) subunit of prolyl 4-hydroxylase, the method comprising administering to a subject in need an effective amount of the pharmaceutical composition of claim 20.

26. A method for treating or preventing a disorder associated with increased expression or activity of the alpha(III) subunit of prolyl 4-hydroxylase, the method comprising administering to a subject in need an effective amount of the antagonist of claim 23.

27. A method of aiding in the diagnosis of a condition associated with altered expression of an alpha(III) subunit of prolyl 4-hydroxylase, comprising (a) detecting the expression of an alpha(III) subunit gene in a test sample;
and
(b) comparing result of step (a) to expression levels of the alpha(III) subunit gene in a control cell, wherein altered of the alpha(III)subunit gene in the test sample is indicative of the condition.

28. A solid phase support comprising the polynucleotide of claim 2.


SEQUENCE LISTING (10 sequences)

SEQ ID NO: 1 - DNA, 2252 bp, human
  misc_feature (1798): where n is A, T, G, or C
atgggtcctggggcgcggctggcggcgctgctggcggtgctggcgctcgggacaggagacccagaaagggctgcggctcggggcgacacgttctcggcgctgaccagcgtggcgcgcgccctggcgcccgagcgccggctgctggggctgctgaggcggtacctgcgcggggaggaggcgcggctgcgggacctgactagattctacgacaaggtactttctttgcatgaggattcaacaacccctgtggctaaccctctgcttgcatttactctcatcaaacgcctgcagtctgactggaggaatgtggtacatagtctggaggccagtgagaacatccgagctctgaaggatggctatgagaaggtggagcaagaccttccagcctttgaggaccttgagggagcagcaagggccctgatgcggctgcaggacgtgtacatgctcaatgtgaaaggcctggcccgaggtgtctttcagagagtcactggctctgccatcactgacctgtacagccccaaacggctcttttctctcacaggggatgactgcttccaagttggcaaggtggcctatgacatgggggattattaccatgccattccatggctggaggaggctgtcagtctcttccgaggatcttacggagagtggaagacagaggatgaggcaagtctagaagatgccttggatcacttggcctttgcttatttccgggcaggaaatgtttcgtgtgccctcagcctctctcgggagtttcttctctacagcccagataataagaggatggccaggaatgtcttgaaatatgaaaggctcttggcagagagccccaaccacgtggtagctgaggctgtcatccagaggcccaatataccccacctgcagaccagagacacctacgaggggctatgtcagaccctgggttcccagcccactctctaccagatccctagcctctactgttcctatgagaccaattccaacgcctacctgctgctccagcccatccggaaggaggtcatccacctggagccctacattgctctctaccatgacttcgtcagtgactcagaggctcagaaaattagagaacttgcagaaccatggctacagaggtcagtggtggcatcaggggagaagcagttacaagtggagtaccgcatcagcaaaagtgcctggctgaaggacactgttgacccaaaactggtgaccctcaaccaccgcattgctgccctcacaggccttgatgtccggcctccctatgcagagtatctgcaggtggtgaactatggcatcggaggacactatgagcctcactttgaccatgctacgtcaccaagcagccccctctacagaatgaagtcaggaaaccgagttgcaacatttatgatctatctgagctcggtggaagctggaggagccacagccttcatctatgccaacctcagcgtgcctgtggttaggaatgcagcactgttttggtggaacctgcacaggagtggtgaaggggacagtgacacacttcatgctggctgtcctgtcctggtgggagataagtgggtggccaacaagtggatacatgagtatggacaggaattccgcagaccctgcagctccagccctgaagactgaactgttggcagagagaagctggtggagtcctgtggctttccagagaagccaggagccaaaagctggggtaggagaggagaaagcatagcagcctcctggaagaaggccttgtcagctttgtctgtgcctcgcaaatcagaggcaagggagaggttgttaccangggacactgagaatgtacatttgatctgccccagccacggaagtcagagtaggatgcacagtacaaaggaggggggagtggaggcctgagagggaagtttctggagttcagatactctctgttgggaacaggacatctcaacagtctcaggttcgatcagtgggtcttttggcactttgaaccttgaccacagggaccaagaagtggcaatgaggacacctgcaggaggggctagcctgactcccagaactttaagactttctccccactgccttctgctgcagcccaagcagggagtgtccccctcccagaagcatatcccagatgagtggtacattatataaggattttttttaagttgaaaacaactttcttttctttttgtatgatggttttttaacacagtcattaaaaatgtttataaatcgaaaaaaaaaaaaaaaaaaaaaaaaaa

SEQ ID NO: 2 - protein, 544 aa, human
MGPGARLAALLAVLALGTGDPERAAARGDTFSALTSVARALAPERRLLGLLRRYLRGEEARLRDLTRFYDKVLSLHEDSTTPVANPLLAFTLIKRLQSDWRNVVHSLEASENIRALKDGYEKVEQDLPAFEDLEGAARALMRLQDVYMLNVKGLARGVFQRVTGSAITDLYSPKRLFSLTGDDCFQVGKVAYDMGDYYHAIPWLEEAVSLFRGSYGEWKTEDEASLEDALDHLAFAYFRAGNVSCALSLSREFLLYSPDNKRMARNVLKYERLLAESPNHVVAEAVIQRPNIPHLQTRDTYEGLCQTLGSQPTLYQIPSLYCSYETNSNAYLLLQPIRKEVIHLEPYIALYHDFVSDSEAQKIRELAEPWLQRSVVASGEKQLQVEYRISKSAWLKDTVDPKLVTLNHRIAALTGLDVRPPYAEYLQVVNYGIGGHYEPHFDHATSPSSPLYRMKSGNRVATFMIYLSSVEAGGATAFIYANLSVPVVRNAALFWWNLHRSGEGDSDTLHAGCPVLVGDKWVANKWIHEYGQEFRRPCSSSPED

SEQ ID NO: 3 - DNA, 27 bp, Artificial Sequence
  Description of Artificial Sequence primer alpha3-r1
gttaggaatgcagcactgttttggtgg

SEQ ID NO: 4 - DNA, 24 bp, Artificial Sequence
  Description of Artificial Sequence primer alpha3-r2
gctggagctgcagggtctgcggaa

SEQ ID NO: 5 - DNA, 30 bp, Artificial Sequence
  Description of Artificial Sequence primer alpha3-r10
aatggcatggtaataatcccccatgctata

SEQ ID NO: 6 - DNA, 22 bp, Artificial Sequence
  Description of Artificial Sequence primer alpha3-1
gttaggaatgcagcactgtttt

SEQ ID NO: 7 - DNA, 18 bp, Artificial Sequence
  Description of Artificial Sequence primer alpha3-2
gctggagctgcagggtct

SEQ ID NO: 8 - DNA, 23 bp, Artificial Sequence
  Description of Artificial Sequence primer alpha3-16
atgggtcctggggcgcggctggc

SEQ ID NO: 9 - DNA, 24 bp, Artificial Sequence
  Description of Artificial Sequence primer alpha3-19
ccgcgcctcctccccgcgcaggta

SEQ ID NO: 10 - DNA, 24 bp, Artificial Sequence
  Description of Artificial Sequence primer alpha3-17
ctgcgggacctgactagattctac